(19) United States
(12) Patent Application Publication
(10) Pub. No.: US 2012/0289420 A1
(43) Pub. Date: Nov. 15, 2012

(54) MICRORNA BIOMARKERS FOR AIRWAY DISEASES

(75) Inventors: SHYAM S. MOHAPATRA, Lutz, FL (US); JIA-WANG WANG, Tampa, FL (US)

(73) Assignee: UNIVERSITY OF SOUTH FLORIDA, Tampa, FL (US)

(21) Appl. No.: 13/424,253

(22) Filed: Mar. 19, 2012

Related U.S. Application Data
(60) Provisional application No. 61/454,447, filed on Mar. 18, 2011.

(51) Int. Cl. C40B 30/04; C07H 21/02; C40B 40/06

(52) U.S. Cl. 506/9; 506/16; 536/24.5; 536/23.1

(57) ABSTRACT

The present invention provides miRNA biomarkers useful for diagnosis, prognosis, and/or treatment of airway diseases such as asthma, asthma exacerbation and nasal polyps.

### CROSS-REFERENCE TO RELATED APPLICATION

The present application claims the benefit of U.S. Provisional Application Ser. No. 61/454,447 filed Mar. 18, 2011, which is hereby incorporated by reference herein in its entirety, including any figures, tables, or drawings.

### BACKGROUND OF THE INVENTION

Asthma is a chronic inflammatory disease of the lungs, characterized by reversible airway hyper-reactivity, excess mucus, and airflow obstruction. Asthma can be triggered and exacerbated by a variety of factors, including more than 200 environmental allergens or irritants, exercise, respiratory tract infections, and co-morbid conditions. Asthma is highly variable over time and among individual patients in many aspects, such as clinical presentation, underlying pathogenic mechanisms, and responsiveness to triggers and therapies.

Diagnosis and treatment of asthma can be difficult. For example, asthma is wrongly diagnosed in many adults as bronchitis or chronic cough. Asthma is also frequently underdiagnosed in childhood. These undiagnosed patients do not receive adequate therapy. Therefore, there is a pressing need for developing noninvasive, sensitive and specific biomarkers for asthma diagnosis.

Epidemiological studies indicate that severe respiratory tract viral infections and repeated allergen exposure may act synergistically and lead to the development of asthma. During pathogenic invasion, the immune response requires rapid, systemic and highly coordinated regulation of a variety of genes to mount an effective defense that identifies and eliminates the invading pathogens.

The miRNA regulatory network plays a critical role in gene regulation during inflammatory responses. First, miRNA-mediated gene regulation is generally faster than other epigenetic mechanisms (such as DNA methylation, histone modification and chromatin alteration) that require transcription. In addition, miRNAs can be secreted into the blood stream from one cell and delivered into other neighboring or remote cells via the exosome pathway (A. Zomer et al., Commun Integr Biol 3, 447, 2010); therefore, a single miRNA can regulate the expression levels of hundreds or even thousands of genes at the accuracy of a single base. As a result, deregulation of miRNAs can have a profound systemic effect on the body.

It is recognized that asthma is a complex disorder of the immune system resulting from the interactions between genetic predisposition and environmental factors. It is also suggested that such interactions are mediated by epigenetics (S. M. Ho, J Allergy Clin Immunol 126, 453, 2010. The present inventors discovered that pathogens (especially viruses)-mediated deregulation of miRNAs can cause the innate immune system to inappropriately sense allergens as pathogens and respond defensively, leading to the genesis and persistence of asthma (Zomer et al. 2010). However, it remains unclear how these interactions correlate to the pathogenesis and progression of asthma and other airway disorders.

Although multiple genetic loci and more than 100 genes have been identified and inferred to contribute to asthma, there is a lack of evidence showing that these genes are not consistently associated with asthma. In addition, none of these genes are consistently associated with identical asthma phenotypes. This is because environmental factors also play a significant role in asthma. The predominance of environmental factors in the etiology of asthma can be evidenced by, for example, the sharp increase in asthma prevalence over the past three decades, the significant variations among populations with a similar racial background but different environmental exposures, and the marked increase in occupational asthma. These phenomena cannot be explained by genetic changes as they take many generations to develop.

MicroRNAs (miRNAs), on average ˜22 nucleotides in length, are non-coding RNAs that primarily target transcriptional regulators and use the Watson-Crick base pairing to specifically inhibit translation. It is reported that deregulation of miRNAs contributes to at least some diseases. For instance, aberrant miRNA expression is a hallmark of tumor development and is associated with the initiation and progression of cancer. miRNA expression profiles have also been used to classify solid and hematologic human cancers, including poorly differentiated tumors and different cell lineages. Studies also suggested that miRNAs can be used as robust biomarkers for diagnosis, staging, prognosis, and evaluation of response to therapy in certain diseases. However, microRNA biomarkers useful for diagnosis, prognosis, and treatment of airway diseases including asthma have not been identified.

### BRIEF SUMMARY OF THE INVENTION

The present invention provides miRNA biomarkers useful for diagnosis, prognosis, and/or treatment of airway diseases such as asthma, asthma exacerbation, and nasal polyps.

In one embodiment, the biomarkers of the present invention are selected from the biomarkers listed in Tables 1-10 and FIGS. 1-7. The expression profile of an individual or a combination of miRNA biomarkers disclosed herein can be used in accordance with the present invention. In some embodiments, one or a combination of biomarkers can be used for diagnosis, prognosis, and treatment of airway diseases. In some embodiments, the airway disease is an upper airway disease such as asthma, asthma exacerbation, nasal polyps, and sinusitis.

### BRIEF DESCRIPTION OF THE DRAWINGS

FIG. 1 shows fold change (Asthma/Normal) of normalized relative human plasma miRNA expression levels. The miRNAs up-regulated in asthma, compared to normal controls, include miR-705, miR-575, let-7d, miR-173p, miR-423-5p, miR-611, miR-674, let-7f-1, miR-23b, miR-223, miR-142-3p, let-7c, miR-25, miR-15b, let-7g, miR-542-5p, and miR-370. The miRNAs down-regulated in asthma, compared to normal controls, include miR-325, mmu-miR-134, miR-198, miR-721, miR-515-3p, miR-680, miR-601, mmu-miR-206, miR-202, miR-671, mmu-miR-381, hsa-miR-630, miR-759, miR-564, miR-709, miR-513, and mmu-miR-298.

FIG. 2 shows relative expression levels of plasma miRNAs from asthma patients (the right bar) and normal volunteers (the left bar).

FIG. 3 shows fold change (Young male/Old Male) of normalized relative human plasma miRNA expression levels.

FIG. 4 shows fold change (Young female/Old female) of normalized relative human plasma miRNA expression levels.

FIG. 5 shows comparison of plasma miRNA expression levels in four groups of asthma patients with exacerbation. This Figure shows miRNAs up-regulated in each group and miRNAs up-regulated in two groups are shown. Arrow points to the group with up-regulation of the miRNA biomarker(s).

FIG. 6 shows fold change (Nasal Polyps/Nasal Epithelial Cells) of Normalized Relative Human miRNA Expression Levels.

FIG. 7 shows levels of individual human nasal polyps miRNAs measured by RT-qPCR miRNA Taqman assay. The individual miRNAs (let-7a, miR-191, miR-21 and miR-494) that have differential expression levels in nasal polyps and normal nasal epithelial cells were selected based on the RT-qPCR miRNA Taqman array assay described above (FIG. 6). The relative expression levels were normalized to an endogenous reference gene U6 small RNA that was not changed in both groups. T-test results are shown above the figures. Each dot represents the expression level from on individual.

### DETAILED DISCLOSURE OF THE INVENTION

The present invention provides miRNA biomarkers useful for diagnosis, prognosis, and treatment of airway diseases such as asthma, sinusitis and nasal polyps. Advantageously, the methods of the present invention are noninvasive, highly specific, and sensitive.

In one embodiment, the present invention profiles circulating miRNA as biomarkers for diagnosis and prognosis of asthma. In another embodiment, the present invention provides microRNA biomarkers for diagnosis and prognosis of nasal polyps.

The present invention also provides treatment methods for airway diseases such as asthma, sinusitis and nasal polyps. Also provided are uses of microRNA biomarker expression profiles of the present invention as targets for screening for therapeutic agents that are useful for treatment of airway diseases such as asthma and sinusitis. In some embodiments, miRNA expression profiles are used to monitor a patient's response to treatment of an airway disease, such as asthma, sinusitis, and nasal polyps.

In one embodiment, the present invention identifies plasma or tissue microRNAs (miRNA) profiles as biomarkers for the detection and prognosis of airway diseases such as asthma and sinusitis. MiRNAs are key epigenetic regulators of gene expression, and their expression is highly regulated. Therefore, deregulation of miRNAs can play an important role in the pathogenesis of various airway diseases such as asthma.

The inventors have determined that miRNAs are directly involved in the pathogenesis of asthma, and their expression pattern in plasma can be associated with the pathophysiological status of asthma. It was discovered that miRNA expression pattern in asthmatic mice (in an ovalbumin-induced asthma model) is distinctly different from that of normal controls. Plasma miRNAs are stable under harsh conditions, including freezing and thawing, high temperature storage (up to 37° C.), acidic conditions, and RNase digestion.

U.S. patent publication US 2010/0286249 A1 (Mohapatra S. S. et al.; application Ser. No. 12/600,803), entitled “Micro-RNAs Modulating Immunity and Inflammation”, published Nov. 11, 2010, is incorporated herein by reference in its entirety. Wang J.-W. et al., “Regulating the Regulators: microRNA and Asthma,” World Allergy Organization Journal, June 2011, 4(6):94-103, which is incorporated herein by reference in its entirety.

miRNA Biomarkers for Upper Airway Diseases

One aspect of the present invention provides microRNA biomarkers useful for diagnosis, prognosis, and treatment of airway diseases such as asthma, nasal polyps and sinusitis. In one embodiment, the present invention provides miRNA biomarkers that are differentially expressed, such as up-regulated, down-regulated, or disregulated in an airway disease, as compared to normal populations that do not have the upper airway disease.

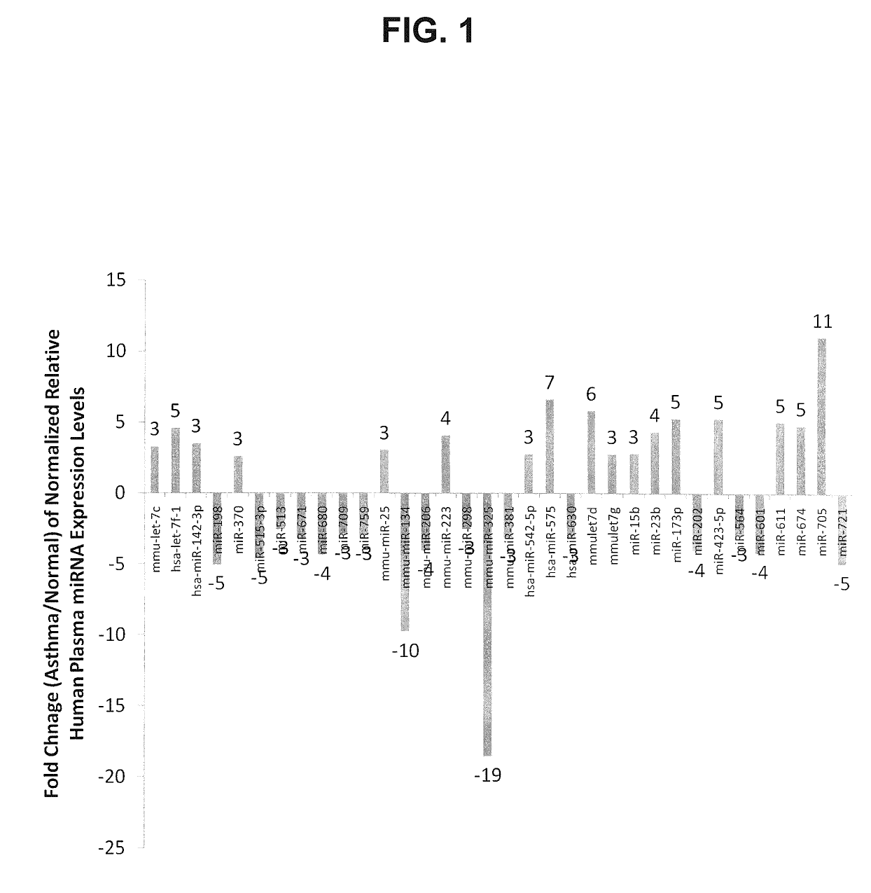

In one embodiment, the biomarkers of the present invention are selected from one or more of the biomarkers listed in Tables 1-10 and FIGS. 1-7 herein. The miRNA expression profile of an individual or a combination of miRNA biomarkers can be used in accordance with the present invention. In some embodiments, one or a combination of biomarkers can be used for diagnosis, prognosis, and treatment of airway diseases such as asthma, nasal polyps and sinusitis.

Table 1 shows relative plasma miRNA expression levels of human plasma miRNAs pooled from five normal volunteers. The miRNA levels are measured by the RT-qPCR miRNA Taqman assay developed by the present inventors. The results show this miRNA profiling assay can sensitively and specifically detect 88 out of 94 miRNAs (those miRNAs have high expression levels in mouse spleens from the dot blot array results) in just 0.8 ml of plasma for each miRNA. The relative expression levels were normalized to mean average. Positive numbers denote expression levels above average, while negative numbers denote expression levels below average. “-” denotes undetected.

| miRNAs | Relative Levels |
|---|---|
| mmu-let-7a | 2.65 |
| mmu-let-7c | −1.81 |
| hsa-let-7f-1 | −3.00 |
| hsa-miR-142-3p | 5.32 |
| miR-15a | 3.49 |
| miR-27a | 52.59 |
| miR-29a | −1.16 |
| miR-150 | 2.47 |
| miR-198 | 1.64 |
| miR-214 | 4.39 |
| miR-295 | −5.10 |
| miR-301b | 1.04 |
| miR-320 | −1.17 |
| miR-370 | −7.43 |
| miR-470 | −2.82 |
| miR-490 | 8.09 |
| miR-494 | 1.46 |
| miR-505 | −5.47 |
| miR-515-3p | 2.24 |
| miR-513 | −13.32 |
| miR-584 | −3.61 |
| miR-588 | −13.81 |
| miR-671 | 1.61 |
| miR-680 | 227.11 |
| miR-690 | −1.95 |
| miR-709 | 8.70 |
| miR-711 | −25.93 |
| miR-759 | −15.75 |
| miR-760 | −1.18 |
| miR-765 | −1.01 |
| hsa-miR-518c* | −2.56 |
| hsa-miR-363* | −2.32 |
| mmu-miR-17 | 1.80 |
| hsa-miR-20a | 2.79 |
| mmu-miR-25 | −1.63 |
| mmu-miR-30d | −2.48 |
| mmu-miR-134 | 59.54 |
| mmu-miR-185 | −1.40 |
| mmu-miR-191 | 2.55 |
| mmu-miR-206 | 115.26 |
| mmu-miR-223 | 9.36 |
| mmu-miR-298 | 1.61 |
| mmu-miR-325 | −5.47 |
| mmu-miR-326 | −3.27 |
| mmu-miR-340-5p | −20.89 |
| mmu-miR-381 | −1.33 |
| mmu-miR-466a-5p | — |
| hsa-miR-493 | 22.06 |
| hsa-miR-527 | −8.05 |
| hsa-miR-542-5p | −9.70 |
| hsa-miR-575 | 2.52 |
| hsa-miR-625 | −4.27 |
| mmu-miR-652 | 1.19 |
| hsa-miR-630 | −1.42 |
| hsa-miR-766 | −1.01 |
| mmulet7d | −3.74 |
| mmulet7g | −1.53 |
| mmulet7i | 21.96 |
| miR-15b | 1.40 |
| miR-21 | 5.19 |
| miR-23b | 1.44 |
| miR-92 | 22.80 |
| miR-173p | −36.65 |
| miR-202 | 5.65 |
| miR-202* | — |
| miR-302 | −3.30 |
| miR-302b | −1.67 |
| miR-328 | 1.21 |
| miR-422b | 18.78 |
| miR-423 | −10.03 |
| miR-540 | 147.73 |
| miR-551a | −3.60 |
| miR-564 | 39.65 |
| miR-573 | −89.73 |
| miR-577 | 1.09 |
| miR-601 | 3.66 |
| miR-608 | 1.66 |
| miR-610 | 2.67 |
| miR-611 | −95.42 |
| miR-637 | 2.50 |
| miR-638 | 2.23 |
| miR-649 | −14.50 |
| miR-658 | 3.67 |
| miR-659 | 1.07 |
| miR-673 | 3.25 |
| miR-674 | −57.22 |
| miR-702 | 2.27 |
| miR-705 | −19.80 |
| miR-710 | −1.89 |
| miR-714 | 5.38 |
| miR-721 | — |
| miR-762 | −2.84 |
| miR-768-5p | −1.02 |
| miR-770-3p | 86.51 |

Table 2 shows the fold change of plasma miRNA expression levels in asthma patients, when compared to normal volunteers. Positive numbers denote that the miRNA expression is up-regulated in asthma, while negative numbers denote that the miRNA expression is down-regulated in asthma. “-” denotes undetected.

| miRNAs | Ratios(Asthma/Normal) |
|---|---|
| mmu-let-7a | 2 |
| mmu-let-7c | 3 |
| hsa-let-7f-1 | 5 |
| hsa-miR-142-3p | 3 |
| miR-15a | −1 |
| miR-27a | −2 |
| miR-29a | 1 |
| miR-150 | −1 |
| miR-198 | −5 |
| miR-214 | — |
| miR-295 | −1 |
| miR-301b | −2 |
| miR-320 | 2 |
| miR-370 | 3 |
| miR-470 | −2 |
| miR-490 | −1 |
| miR-494 | −1 |
| miR-505 | −2 |
| miR-515-3p | −5 |
| miR-513 | −3 |
| miR-584 | 1 |
| miR-588 | −2 |
| miR-671 | −3 |
| miR-680 | −4 |
| miR-690 | 1 |
| miR-709 | −3 |
| miR-711 | 2 |
| miR-759 | −3 |
| miR-760 | −2 |
| miR-765 | −1 |
| miR-hsa-miR-518c* | — |
| miR-hsa-miR-363* | — |
| mmu-miR-17 | — |
| hsa-miR-20a | 2 |
| mmu-miR-25 | 3 |
| mmu-miR-30d | −2 |
| mmu-miR-134 | −10 |
| mmu-miR-185 | 2 |
| mmu-miR-191 | −1 |
| mmu-miR-206 | −4 |
| mmu-miR-223 | 4 |
| mmu-miR-298 | −3 |
| mmu-miR-325 | −19 |
| mmu-miR-326 | 2 |
| mmu-miR-340-5p | — |
| mmu-miR-381 | −3 |
| mmu-miR-466a-5p | — |
| hsa-miR-493 | −2 |
| hsa-miR-527 | — |
| hsa-miR-542-5p | 3 |
| hsa-miR-575 | 7 |
| hsa-miR-625 | −2 |
| mmu-miR-652 | −2 |
| hsa-miR-630 | −3 |
| hsa-miR-766 | — |
| mmulet7d | 6 |
| mmulet7g | 3 |
| mmulet7i | −2 |
| miR-15b | 3 |
| miR-21 | 2 |
| miR-23b | 4 |
| miR-92 | −2 |
| miR-173p | 5 |
| miR-202 | −4 |
| miR-202* | — |
| miR-302 | −2 |
| miR-302b | — |
| miR-328 | −1 |
| miR-422b | −1 |
| miR-423-5p | 5 |
| miR-540 | −1 |
| miR-551a | −2 |
| miR-564 | −3 |
| miR-573 | −1 |
| miR-577 | −2 |
| miR-601 | −4 |
| miR-608 | 1 |
| miR-610 | −1 |
| miR-611 | 5 |
| miR-637 | 1 |
| miR-638 | 2 |
| miR-649 | −2 |
| miR-658 | −2 |
| miR-659 | 1 |
| miR-673 | −1 |
| miR-674 | 5 |
| miR-702 | −1 |
| miR-705 | 11 |
| miR-710 | −3 |
| miR-714 | −2 |
| miR-721 | −5 |
| miR-762 | 2 |
| miR-768-5p | 2 |
| miR-770-3p | −2 |

Table 3 shows the enriched pathways targeted by the miRNAs up-regulated in asthma. The up-regulated miRNA biomarkers include miR-705, miR-575, let-7d, miR-173p, miR-423-5p, miR-611, miR-674, let-7f-1, miR-23b, miR-223, miR-142-3p, let-7c, miR-25, miR-15b, let-7g, miR-542-5p, and miR-370, which target enriched pathways including the MAPK signaling pathway, TGF-beta signaling pathway and ErbB signaling pathway.

| KEGG Pathway | # of Genes | −In(p-value) |
|---|---|---|
| MAPK signaling pathway | 100 | 33.28 |
| Focal adhesion | 76 | 24.48 |
| Axon guidance | 54 | 22.34 |
| Regulation of actin | 76 | 19.6 |
| cytoskeleton | | |
| TGF-beta signaling pathway | 35 | 10.43 |
| Oxidative phosphorylation | 6 | 10.12 |
| ErbB signaling pathway | 34 | 9.97 |
| Adherens junction | 29 | 9.41 |
| Wnt signaling pathway | 49 | 9.26 |
| mTOR signaling pathway | 21 | 8.77 |
| Long-term depression | 29 | 8.69 |
| GnRH signaling pathway | 34 | 7.81 |
| Fc epsilon RI signaling | 28 | 7.27 |
| pathway | | |
| D-Glutamine and D- | 4 | 6.75 |
| glutamate metabolism | | |
| Tryptophan metabolism | 2 | 6.48 |
| Gap junction | 32 | 6.23 |
| Ubiquitin mediated | 41 | 6.09 |
| proteolysis | | |
| VEGF signaling pathway | 25 | 5.69 |
| Toll-like receptor signaling | 33 | 5.48 |
| pathway | | |
| SNARE interactions in | 15 | 5.31 |
| vesicular transport | | |
| p53 signaling pathway | 24 | 5.28 |
| Calcium signaling pathway | 48 | 5.06 |
| ECM-receptor interaction | 27 | 5 |
| Starch and sucrose | 5 | 4.87 |
| metabolism | | |
| Arachidonic acid metabolism | 3 | 4.86 |
| Folate biosynthesis | 1 | 4.56 |
| Pyruvate metabolism | 2 | 4.19 |
| Pyrimidine metabolism | 8 | 4.18 |
| Dorso-ventral axis formation | 11 | 4.11 |
| Tight junction | 39 | 4.07 |
| Insulin signaling pathway | 40 | 4.07 |
| Glycolysis/Gluconeogenesis | 5 | 3.96 |
| T cell receptor signaling | 28 | 3.92 |
| pathway | | |
| Long-term potentiation | 21 | 3.85 |
| Jak-STAT signaling pathway | 42 | 3.39 |
| Bile acid biosynthesis | 2 | 3.33 |
| Melanogenesis | 29 | 3.08 |
| Complement and coagulation | 7 | 3.02 |
| cascades | | |
| Asthma | 2 | 2.05 |

Table 4 shows the enriched pathways targeted by the miRNAs down-regulated in asthma. The down-regulated miRNA biomarkers include miR-325, mmu-miR-134, miR-198, miR-721, miR-515-3p, miR-680, miR-601, mmu-miR-206, miR-202, miR-671, mmu-miR-381, hsa-miR-630, miR-759, miR-564, miR-709, miR-513, and mmu-miR-298.

| KEGG Pathway | # of Genes | −In(p-value) |
|---|---|---|
| Adherens junction | 13 | 11.29 |
| TGF-beta signaling pathway | 13 | 7.61 |
| MAPK signaling pathway | 24 | 5.28 |
| mTOR signaling pathway | 7 | 4.16 |
| Glycan structures - biosynthesis 1 | 13 | 4.1 |
| Wnt signaling pathway | 15 | 4.09 |
| Focal adhesion | 18 | 3.74 |
| Regulation of actin cytoskeleton | 19 | 3.7 |
| Cell cycle | 12 | 3.54 |
| Hedgehog signaling pathway | 7 | 3.14 |

Table 5 shows the fold change of plasma miRNA expression levels in male asthma patients with exacerbation (young v. old). Relative plasma miRNA expression levels of human plasma miRNAs pooled from 7 young male patients (23 to 48 years old) and 5 old male patients (56 to 71 years old) are measured by the RT-qPCR miRNA Taqman assay.

| miRNAs | Ratios(Young male/Old male) |
|---|---|
| let-7a | −2 |
| let-7b | −1 |
| let-7e | 2 |
| let-7g | −1 |
| miR-101b | −1 |
| miR-15b | 16 |
| miR-1937a | 2 |
| miR-29a | −1 |
| miR-669i | 2 |
| miR-185 | −2 |
| let-7c | −2 |
| let-7d | 3 |
| let-7f | — |
| let-7i | −1 |
| miR-1 | — |
| miR-206 | −1 |
| miR-17 | 1 |
| miR-134 | 1 |
| miR-15a | 2 |
| miR-709 | 2 |
| miR-27a | −1 |
| miR-302a | −1 |
| miR-302b | −1 |
| miR-20a | 3 |
| miR-466a-5p | −4 |
| miR-721(/1000) | 3 |
| miR-328 | −1 |
| miR-674 | 1 |
| miR-30d | 3 |
| miR-494 | 2 |
| miR-142-3p | 1 |
| miR-470 | 1 |
| miR-23b | −1 |
| miR-223 | 1 |
| miR-25 | −1 |
| miR-150 | −1 |
| miR-191 | −1 |
| miR-705 | −2 |
| miR-673-5p | −2 |
| miR-714 | −1 |
| miR-680 | −1 |
| miR-490-3p | −1 |
| miR-760-3p | 1 |
| miR-770-3p | −1 |
| miR-21 | 1 |
| miR-370 | −1 |
| miR-378 | −2 |
| miR-671-5p | — |
| miR-759 | 1 |
| miR-214 | — |
| miR-295 | — |
| miR-296-5p | 1 |
| miR-340-5p | — |
| miR-423-5p | −13 |
| miR-540-3p | −1 |
| miR-652 | — |
| miR-702 | −1 |
| miR-710 | −1 |
| miR-320(/1000) | 3 |
| miR-381 | 2 |
| miR-690 | 2 |
| miR-611 | −2 |
| miR-659 | −1 |
| miR-513a-5p | −1 |
| miR-518a-5p | −1 |
| miR-601 | −1 |
| miR-584 | −2 |
| miR-637 | −2 |
| miR-575(/10) | −1 |
| miR-325 | 2 |
| miR-608 | −2 |
| miR-515-3p | −1 |
| miR-92a | −2 |
| miR-542-5p | −1 |
| miR-610 | −1 |
| miR-301b | 2 |
| miR-573 | −2 |
| miR-765 | −2 |
| miR-766 | −1 |
| miR-762 | −4 |
| miR-638 | −2 |
| miR-625 | 1 |
| miR-298 | −1 |
| miR-630 | −1 |
| miR-198 | −1 |
| miR-658 | −2 |
| miR-711 | −4 |
| miR-493 | 1 |
| miR-202 | 1 |
| miR-588 | 2 |
| miR-577 | 2 |
| miR-505 | −2 |
| miR-649 | 1 |
| miR-564 | −1 |
| miR-551a | −1 |
| miR-326 | −1 |

Table 6 shows the fold change of plasma miRNA expression levels in female asthma patients with exacerbation (young v. old). Relative plasma miRNA expression levels of human plasma miRNAs pooled from 11 young female patients (25 to 47 years old) and 6 old female patients (58 to years old) are measured by the RT-qPCR miRNA Taqman assay.

| miRNAs | Ratios(Young female/Old female) |
|---|---|
| let-7a | −1 |
| let-7b | −1 |
| let-7e | 1 |
| let-7g | −1 |
| miR-101b | −1 |
| miR-15b | −8 |
| miR-1937a | −2 |
| miR-29a | −1 |
| miR-669i | 2 |
| miR-185 | 1 |
| let-7c | −1 |
| let-7d | −1 |
| let-7f | 3 |
| let-7i | −14 |
| miR-1 | 2 |
| miR-206 | 1 |
| miR-17 | — |
| miR-134 | 2 |
| miR-15a | 1 |
| miR-709 | 2 |
| miR-27a | 1 |
| miR-302a | −1 |
| miR-302b | — |
| miR-20a | −1 |
| miR-466a-5p | 3 |
| miR-721(/1000) | 1 |
| miR-328 | −1 |
| miR-674 | 1 |
| miR-30d | −3 |
| miR-494 | −2 |
| miR-142-3p | 1 |
| miR-470 | 1 |
| miR-23b | −1 |
| miR-223 | 1 |
| miR-25 | — |
| miR-150 | 1 |
| miR-191 | −1 |
| miR-705 | 1 |
| miR-673-5p | −2 |
| miR-714 | −1 |
| miR-680 | −2 |
| miR-490-3p | 1 |
| miR-760-3p | −2 |
| miR-770-3p | 1 |
| miR-21 | 15 |
| miR-370 | 1 |
| miR-378 | −1 |
| miR-671-5p | — |
| miR-759 | −3 |
| miR-214 | 3 |
| miR-295 | — |
| miR-296-5p | −1 |
| miR-340-5p | — |
| miR-423-5p | — |
| miR-540-3p | −1 |
| miR-652 | — |
| miR-702 | 1 |
| miR-710 | −1 |
| miR-320(/1000) | −1 |
| miR-381 | −1 |
| miR-690 | 1 |
| miR-611 | −2 |
| miR-659 | −1 |
| miR-513a-5p | −4 |
| miR-518a-5p | 2 |
| miR-601 | 1 |
| miR-584 | −4 |
| miR-637 | −6 |
| miR-575 | 16 |
| miR-325 | 2 |
| miR-608 | −1 |
| miR-515-3p | 4 |
| miR-92a | 1 |
| miR-542-5p | −2 |
| miR-610 | −5 |
| miR-301b | 1 |
| miR-573 | 2 |
| miR-765 | −2 |
| miR-766 | 1 |
| miR-762 | −3 |
| miR-638 | −2 |
| miR-625 | −1 |
| miR-298 | 1 |
| miR-630 | −1 |
| miR-198 | −1 |
| miR-658 | 1 |
| miR-711 | −2 |
| miR-493 | −2 |
| miR-202 | −1 |
| miR-588 | −1 |
| miR-577 | −1 |
| miR-505 | −2 |
| miR-649 | −1 |
| miR-564 | 1 |
| miR-551a | 1 |
| miR-326 | 2 |

Table 7 shows a comparison of plasma miRNA expression levels in four groups of asthma patients with exacerbation.

| miRNAs | Young female/Old female | Young male/Old male |
|---|---|---|
| *miR-320 | <2 | 3 |
| *miR-721 | <2 | 3 |
| let-7c | <2 | −2 |
| let-7i | −14 | <2 |
| miR-134 | 2 | <2 |
| miR-15b | −8 | 16 |
| miR-185 | <2 | −2 |
| miR-1937a | −2 | 2 |
| miR-20a | <2 | 3 |
| miP-301b | <2 | 2 |
| miR-30d | −3 | 3 |
| miR-325 | 2 | <2 |
| miR-326 | 2 | <2 |
| miR-378 | <2 | −2 |
| miR-381 | <2 | 2 |
| miR-423-5p | <2 | −13 |
| miR-493 | −2 | <2 |
| miR-494 | −2 | 2 |
| miR-513a-5p | −4 | <2 |
| miR-515-3p | 4 | <2 |
| miR-542-5p | −2 | <2 |
| miR-575 | 16 | <2 |
| miR-577 | <2 | 2 |
| miR-584 | 4 | <2 |
| miR-588 | <2 | 2 |
| miR-608 | <2 | 2 |
| miR-610 | −5 | <2 |
| miR-637 | [symbol] | −2 |
| miR-638 | −2 | <2 |
| miR-658 | <2 | −2 |
| miR-669i | [symbol] | [symbol] |
| miR-673-5p | [symbol] | [symbol] |
| miR-680 | −2 | <2 |
| miR-690 | <2 | 2 |
| miR-705 | <2 | −2 |
| miR-709 | [symbol] | [symbol] |
| miR-711 | [symbol] | [symbol] |
| miR-760-3p | −2 | <2 |
| miR-762 | [symbol] | [symbol] |
| miR-765 | [symbol] | [symbol] |
| miR-92a | <2 | −2 |

[symbol]

indicates data missing or illegible when filed

Table 8 shows the enriched pathways targeted by the miRNAs up-regulated in nasal polyps. These miRNAs include miR-765, miR-705, let-7f-1, let7d, miR-494, let-7a, miR-23b, miR-191, and miR-21.

| KEGG Pathway | # of Genes | −In(p-value) |
|---|---|---|
| Focal adhesion | 47 | 14.98 |
| MAPK signaling pathway | 55 | 12.74 |
| Amyotrophic lateral sclerosis (ALS) | 9 | 10.37 |
| Axon guidance | 30 | 9.25 |
| Jak-STAT signaling pathway | 34 | 8.33 |
| Pancreatic cancer | 19 | 6.9 |
| TGF-beta signaling pathway | 22 | 6.88 |
| p53 signaling pathway | 18 | 6.85 |
| Circadian rhythm | 6 | 6.52 |
| Ribosome | 1 | 5.79 |
| Oxidative phosphorylation | 4 | 5.52 |
| Antigen processing and presentation | 1 | 5.05 |
| Prostate cancer | 20 | 4.96 |
| Wnt signaling pathway | 29 | 4.85 |
| Adherens junction | 17 | 4.79 |
| ErbB signaling pathway | 19 | 4.18 |
| Regulation of actin cytoskeleton | 37 | 4.04 |
| Tryptophan metabolism | 1 | 3.98 |
| Metabolism of xenobiotics by | 1 | 3.98 |
| cytochrome P450 | | |
| Tight junction | 26 | 3.96 |
| Starch and sucrose metabolism | 2 | 3.78 |
| ECM-receptor interaction | 17 | 3.47 |
| Heparan sulfate biosynthesis | 6 | 3.33 |
| Glycosylphosphatidylinositol(GPI)- | 6 | 3.33 |
| anchor | | |
| Glycolysis/Gluconeogenesis | 2 | 3.22 |
| T cell receptor signaling pathway | 18 | 3.08 |
| mTOR signaling pathway | 11 | 2.98 |

Table 9 shows the enriched pathways targeted by the miRNAs down-regulated in nasal polyps. These miRNAs include miR-721, miR-295, miR-690, miR-302b, miR-466a-5p, miR-15a, miR-759, miR-202, miR-302, and miR-608.

| GG Pathway | # of Genes | −In(p-value) |
|---|---|---|
| TGF-beta signaling pathway | 25 | 16.22 |
| Wnt signaling pathway | 31 | 11.28 |
| Prostate cancer | 22 | 11.22 |
| Adherens junction | 19 | 10.96 |
| MAPK signaling pathway | 45 | 10.24 |
| Focal adhesion | 36 | 9.35 |
| Regulation of actin cytoskeleton | 37 | 8.35 |
| mTOR signaling pathway | 13 | 8.08 |
| D-Glutamine and D-glutamate metabolism | 3 | 7.25 |
| Axon guidance | 24 | 6.86 |
| Insulin signaling pathway | 25 | 5.82 |
| Cell cycle | 21 | 5.29 |
| Endometrial cancer | 12 | 5.08 |
| Oxidative phosphorylation | 3 | 4.77 |
| T cell receptor signaling pathway | 17 | 4.49 |
| Ubiquitin mediated proteolysis | 22 | 4.17 |
| Polyunsaturated fatty acid biosynthesis | 6 | 3.95 |
| Type II diabetes mellitus | 9 | 3.84 |
| ErbB signaling pathway | 16 | 3.8 |
| Starch and sucrose metabolism | 1 | 3.72 |
| SNARE interactions in vesicular transport | 8 | 3.14 |
| Phosphatidylinositol signaling system | 13 | 3.11 |
| Tryptophan metabolism | 1 | 3.05 |
| Tight junction | 21 | 3.03 |

Table 10 shows fold change of miRNA expression levels in nasal polyps compared to normal nasal epithelial cells. Relative plasma miRNA expression levels measured by RT-qPCR miRNA Taqman assay of human plasma miRNAs pooled from 6 patients and 5 normal volunteers.

| | Ratios(Nasal |
|---|---|
| | polyps/Normal nasal |
| miRNAs | epithelial cells) |
| mmu-let-7a | 2.50557 |
| mmu-let-7c | 1.288195 |
| hsa-let-7f-1 | 2.777389 |
| hsa-miR-142-3p | −2.39661 |
| miR-15a | −2.99313 |
| miR-27a | −1.24058 |
| miR-29a | 1.61698 |
| miR-150 | −1.25228 |
| miR-198 | −1.88815 |
| miR-214 | 1.682037 |
| miR-295 | −6.49557 |
| miR-301b | 1.376651 |
| miR-320 | −1.18042 |
| miR-370 | 1.181571 |
| miR-470 | −1.38755 |
| miR-490 | 1.214341 |
| miR-494 | 2.514489 |
| miR-505 | −1.18351 |
| miR-515-3p | −2.09964 |
| miR-513 | −2.08464 |
| miR-584 | −2.18634 |
| miR-588 | −1.34309 |
| miR-671 | −1.02926 |
| miR-680 | −1.52636 |
| miR-690 | −4.49503 |
| miR-711 | −1.23118 |
| miR-759 | −2.90936 |
| miR-760 | −1.25218 |
| miR-765 | 3.831762 |
| miR-hsa-miR-518c* | −1.30962 |
| miR-hsa-miR-363* | −1.47036 |
| hsa-miR-20a | 1.62959 |
| mmu-miR-25 | 1.826089 |
| mmu-miR-30d | 1.057096 |
| mmu-miR-134 | −1.30118 |
| mmu-miR-185 | 1.269764 |
| mmu-miR-191 | 2.030637 |
| mmu-miR-206 | −1.5731 |
| mmu-miR-223 | −1.01437 |
| mmu-miR-298 | 1.374237 |
| mmu-miR-325 | −1.51755 |
| mmu-miR-326 | 1.40507 |
| mmu-miR-340-5p | 1.091312 |
| mmu-miR-381 | 1.306937 |
| mmu-miR-466a-5p | −3.77081 |
| hsa-mm-493 | 1.244195 |
| hsa-miR-527 | 1.172426 |
| hsa-miR-542-5p | −1.9287 |
| hsa-miR-575 | −1.04002 |
| hsa-miR-625 | −1.38852 |
| mmu-miR-652 | 1.220683 |
| hsa-miR-630 | 1.125366 |
| hsa-miR-766 | 1.467139 |
| mmulet7d | 2.726145 |
| mmulet7g | 2.071226 |
| mmulet7i | 1.552477 |
| miR-15b | 1.57785 |
| miR-21 | 1.684596 |
| miR-23b | 2.160861 |
| miR-173p | 1.912192 |
| miR-202 | −2.76316 |
| miR-202* | — |

| | Ratios(Young |
|---|---|
| miRNAs | male/Old male) |
| miR-302b | −4.09521 |
| miR-328 | 1.231797 |
| miR-422b | 1.121007 |
| miR-423 | 1.758631 |
| miR-540 | −1.29422 |
| miR-551a | 1.126018 |
| miR-564 | −1.62517 |
| miR-573 | 1.401764 |
| miR-577 | — |
| miR-601 | −1.18281 |
| miR-608 | −2.51817 |
| miR-610 | −1.62036 |
| miR-611 | 1.114214 |
| miR-637 | 1.562755 |
| miR-638 | 2.228703 |
| miR-649 | 1.026359 |
| miR-658 | −1.19247 |
| miR-659 | 1.038787 |
| miR-673 | 1.506193 |
| miR-674 | −1.80408 |
| miR-702 | −1.12736 |
| miR-705 | 2.826162 |
| miR-710 | −1.45264 |
| miR-714 | 2.341929 |
| miR-721 | −6.76495 |
| miR-762 | 1.379542 |
| miR-768-5p | 1.560289 |
| miR-770-3p | −1.34124 |
| mmu-miR-17 | 1.38313 |
| miR-302 | −2.75586 |

miRNA Biomarkers for Asthma

In some embodiments, the present invention provides microRNA biomarkers for diagnosis and/or prognosis of asthma, including one or any combination of two or more biomarkers selected from Tables 2-4 and FIGS. 1-4.

In some embodiments, the biomarkers are selected from one or more miRNAs up-regulated or over-expressed in asthma. In some embodiments, the biomarkers for asthma are selected from miR-705, miR-575, let-7d, miR-173p, miR-423-5p, miR-611, miR-674, let-7f-1, miR-23b, miR-223, miR-142-3p, let-7c, miR-25, miR-15b, let-7g, and miR-542-5p, miR-370, or any combination thereof, wherein the up-regulation or over-expression of the one or more aforementioned miRNA biomarkers in the subject's biological sample, when compared to a control, indicates that the subject has asthma.

In some specific embodiments, the biomarkers are selected from one or more miRNAs up-regulated or over-expressed more than 5-fold, 4-fold, 3-fold, 2-fold, or 1-fold in asthma patients, when compared to a control (as shown in FIG. 1 and Table 2). In some embodiments, the biomarkers for asthma are selected from miR-705, miR-575, let-7d, miR-173p, miR-423-5p, miR-611, let-7f-1, or any combination thereof, wherein the up-regulation or over-expression of the one or more aforementioned miRNA biomarkers in the subject's biological sample, when compared to a control, indicates that the subject has asthma.

In some embodiments, the biomarkers are selected from one or more miRNAs down-regulated in asthma. In some embodiments, the biomarkers for asthma are selected from miR-325, miR-134, miR-198, miR-721, miR-515-3p, miR-680, miR-601, miR-206, miR-202, miR-671, miR-381, miR-630, miR-759, miR-564, miR-709, miR-513, and -miR-298, or any combination thereof, wherein the down-regulation of the one or more aforementioned miRNA biomarkers in the subject's biological sample, when compared to a control, indicates that the subject has asthma. In some specific embodiments, the biomarkers are selected one or more miRNAs down-regulated or under-expressed more than 5-folds-folds, 4-folds, 3-folds, 2-folds, or 1-fold in asthma patients, when compared to a control (as shown in FIG. 1 and Table 2). In some embodiments, the biomarkers for asthma are selected from miR-325, miR-134, miR-198, miR-721, miR-515-3p, or any combination thereof, wherein the down-regulation or under-expression of the one or more aforementioned miRNA biomarkers in the subject's biological sample, when compared to a control, indicates that the subject has asthma.

In one embodiment, the biomarkers for asthma are selected from miR-142-3p, let-7c miR-21, or any combination thereof, wherein the up-regulation or over-expression of the one or more aforementioned miRNA biomarkers in the subject's biological sample, when compared to a control, indicates that the subject has asthma.

In one embodiment, the biomarkers for asthma are selected from miR-494, miR-150, or both, wherein the down-regulation or under-expression of one or more of the aforementioned miRNA biomarkers in the subject's biological sample, when compared to a control, indicates that the subject has asthma.

In one embodiment, the present invention provides microRNA biomarkers for asthma, that target pathways shown in Tables 3 and 4, including the MAPK signaling pathway, TGF-beta signaling pathway ErbB signaling pathway, mTOR signaling pathway, and Wnt signaling pathway.

Gender- and Age-Specific miRNA Biomarkers for Asthma

In one embodiment, the present invention provides gender- and age-specific miRNA biomarkers for diagnosis and/or prognosis of asthma.

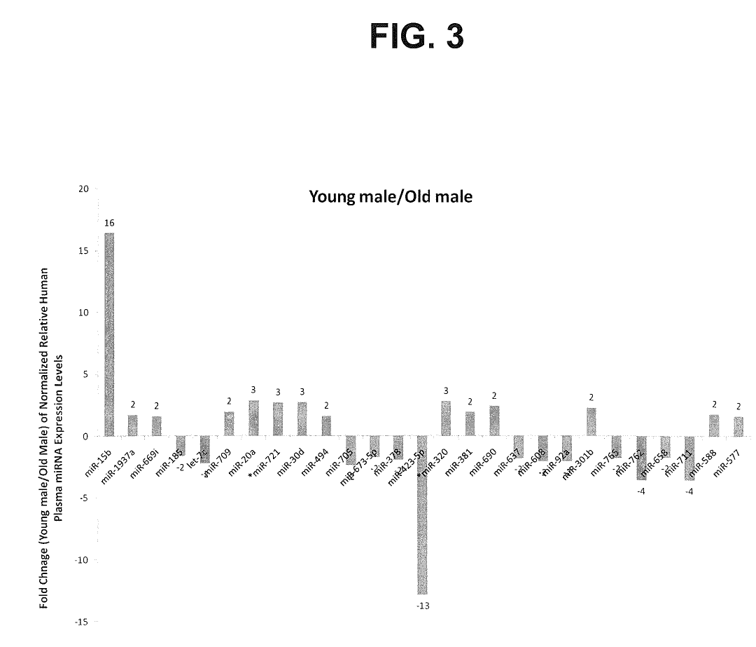

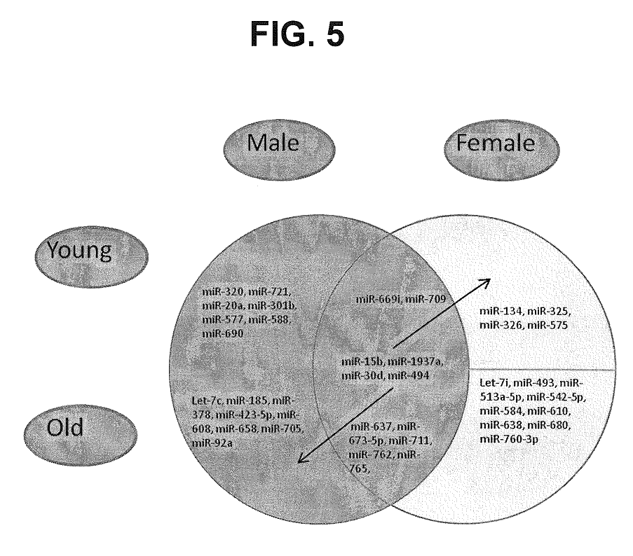

In some embodiments, microRNA biomarkers that are up-regulated or over-expressed in young male patients are shown in Tables 5 and 7 and FIGS. 3 and 5 (biomarkers with young male/old male fold change >3, 2, 1, or 0). In some embodiments, the microRNA biomarkers diagnosing whether a young male has asthma is selected from miR-15b, miR-20a, miR-721, let-7d, miR-30d, miR-320, or any combination thereof, wherein the up-regulation or over-expression of the one or more aforementioned miRNA biomarkers in a young male subject's biological sample, when compared to a control, indicates that the young male subject has asthma.

In an embodiment, the young male subject is a human less than 50-year-old, or of any age younger including, but not limited to, 45-, 40-, 35-, 30-, 25-, 20-, 15-, 10, or 5-year old.

In some embodiments, microRNA biomarkers that are up-regulated or over-expressed in old male patients are shown in Tables 5 and 7 and FIGS. 3 and 5 (biomarkers with young male/old male fold change <−3, −2, −1, or 0). In some embodiments, the microRNA biomarkers diagnosing whether an old male has asthma is selected from miR-423-5p, miR-762, miR-466-5p, miR-711, or any combination thereof, wherein the up-regulation or over-expression of the one or more aforementioned miRNA biomarkers in an old male subject's biological sample, when compared to a control, indicates that the old male subject has asthma. In an embodiment, the old male subject is a human older than 50-year-old, or of any age older including, but not limited to, 55-, 60-, 65-, 70-, 75, or 80-year old.

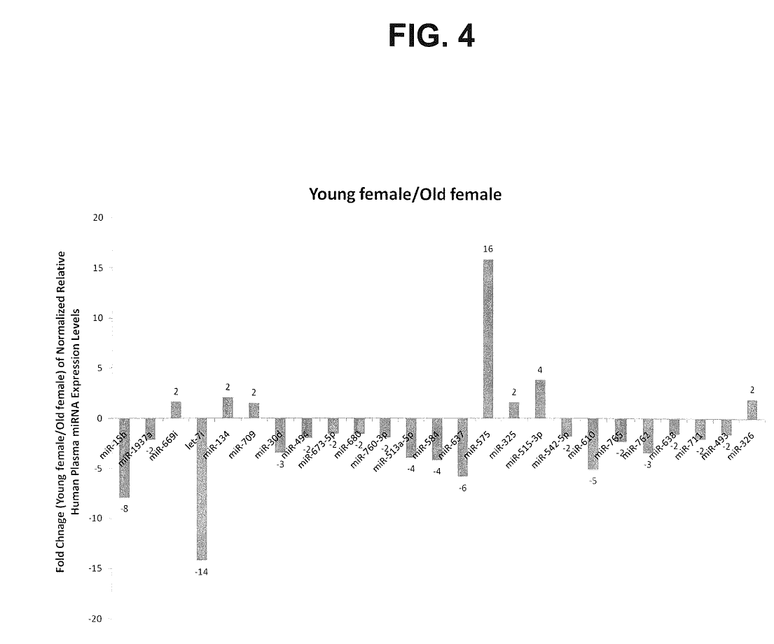

In some embodiments, microRNA biomarkers that are up-regulated or over-expressed in young female patients are shown in Tables 6 and 7 and FIGS. 4 and 5 (biomarkers with young female/old female fold change >3, 2, 1, or 0). In some embodiments, the microRNA biomarkers diagnosing whether a young female has asthma are selected from miR-466a-5p, miR-21, let-7f, miR-214, miR-575, miR-513p, or any combination thereof, wherein the up-regulation or over-expression of the one or more aforementioned miRNA biomarkers in a young female subject's biological sample, when compared to a control, indicates that the young female subject has asthma. In an embodiment, the young female subject is a human less than 50-year-old, or of any age younger including, but not limited to, 45-, 40-, 35-, 30-, 25-, 20-, 15-, 10, or 5-year old.

In some embodiments, microRNA biomarkers that are up-regulated or over-expressed in old female patients are shown in Tables 6 and 7 and FIGS. 4 and 5 (biomarkers with young female/old female fold change <−3, −2, −1, or 0). In some embodiments, the microRNA biomarkers diagnosing whether an old female has asthma is selected from miR-15b, let-7i, miR-30d, miR-759, miR-513a-5p, miR-584, miR-637, miR-610, miR-762, or any combination thereof, wherein the up-regulation or over-expression of the one or more aforementioned miRNA biomarkers in an old female subject's biological sample, when compared to a control, indicates that the old female subject has asthma. In an embodiment, the old female subject is a human older than 50-year-old, or of any age older including, but not limited to, 55-, 60-, 65-, 70-, 75, or 80-year old.

miRNA Biomarkers for Nasal Polyps

In some embodiments, the present invention provides microRNA biomarkers for nasal polyps, including one or any combination of two or more biomarkers selected from Tables 8-10. In some embodiments, the biomarkers are selected from one or more miRNAs up-regulated in nasal polyps. In some embodiments, the biomarkers are selected from miRNAs down-regulated in nasal polyps.

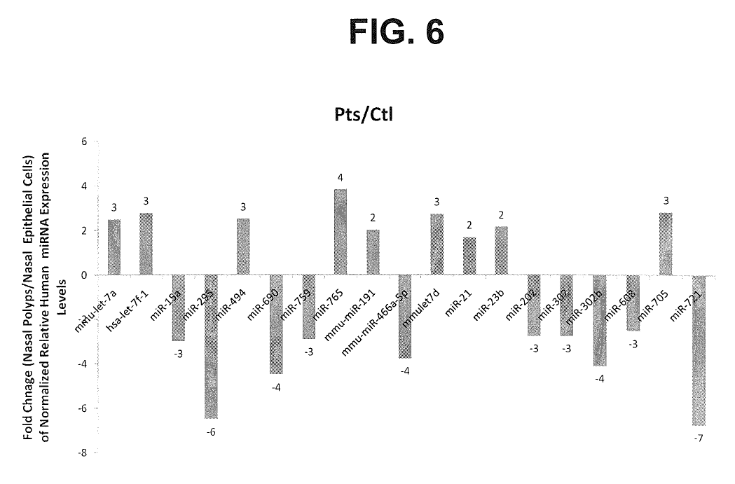

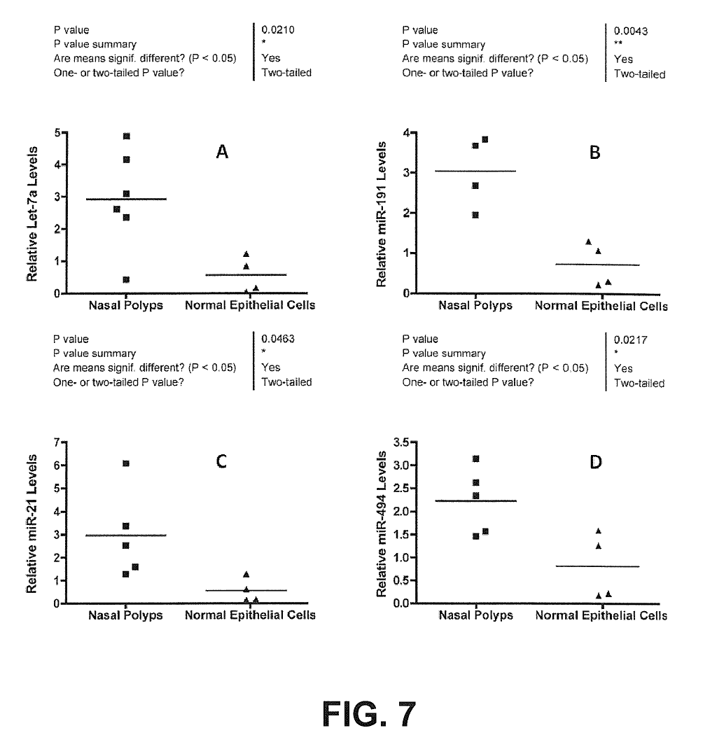

In some specific embodiments, the biomarkers are selected one or more miRNAs up-regulated or over-expressed more than 5-fold, 4-fold, 3-fold, 2-fold, or 1-fold in patients with nasal polyps, when compared to a control (as shown in Table 10 and FIGS. 6 and 7), wherein the over-expression of the one or more aforementioned biomarkers indicate that the subject has a nasal polyp.

In some specific embodiments, the biomarkers are selected one or more miRNAs down-regulated or under-expressed more than 5-fold, 4-fold, 3-fold, 2-fold, or 1-fold in patients with nasal polyps, when compared to a control (as shown in Table 10 and FIGS. 6 and 7), wherein the down-regulation or under-expression of the one or more aforementioned biomarkers indicates that the subject has a nasal polyp. In some embodiments, the miRNAs biomarkers up-regulated in nasal polyps include, but are not limited to, miR-765, miR-705, let-7f-1, let7d, miR-494, let-7a, miR-23b, miR-191, and miR-21. The miRNAs biomarkers down-regulated in nasal polyps include, but are not limited to, miR-721, miR-295, miR-690, miR-302b, miR-466a-5p, miR-15a, miR-759, miR-202, miR-302, and miR-608.

### Diagnosis of Airway Diseases

In one embodiment, the subject invention provides a method for diagnosing and/or prognosis of an airway disease, comprising:

a) determining a miRNA expression profile (expression level) in a biological sample from the subject;

b) characterizing the subject's miRNA profile; and

c) comparing the subject's miRNA profile with the miRNA profile of a control miRNA profile from subjects that do not have the airway disease.

In one embodiment, the method further includes obtaining the biological sample from the subject. In one embodiment, the diagnosis and/or prognosis of airway diseases can be determined by comparing the subject's microRNA profile to a reference miRNA profile, such as one that corresponds to biological samples obtained from a normal population that do not have the airway disease, or that corresponds to biological samples obtained from a population that have the disease. Optionally, the reference profile comprises multiple miRNA expression profiles, with each corresponding to a different airway disease and/or stage of airway disease.

The terms “patient” and “subject” are used interchangeably herein, describing an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human.

The term “biological sample,” as used herein, includes but is not limited to, a sample containing tissues, cells, and/or biological fluids isolated from a subject. Examples of biological samples include but, are not limited to, tissues, cells, biopsies, blood, lymph, serum, plasma, urine, saliva, mucus, and tears. In one embodiment, the biological sample is a blood sample (such as a plasma sample). In one embodiment, the biological sample is a tissue sample from nasal epithelium. A biological sample may be obtained directly from a subject (e.g., by blood or tissue sampling) or from a third party (e.g., received from an intermediary, such as a healthcare provider).

In some embodiments, the present invention provides methods for diagnosing asthma by characterizing one or a combination of biomarkers selected from Tables 2-4 and FIGS. 1-4. In some embodiments, the present invention provides methods for diagnosing asthma by characterizing miRNA selected from miR-705, miR-575, let-7d, miR-173p, miR-423-5p, miR-611, miR-674, let-7f-1, miR-23b, miR-223, miR-142-3p, let-7c, miR-25, miR-15b, let-7g, and miR-542-5p, miR-370, and any combination of two or more of the foregoing, wherein the up-regulation or over-expression of one or more of the aforementioned miRNA, when compared to a control, indicates that the subject has asthma.

In some embodiments, the present invention provides methods for diagnosing asthma by characterizing miRNA selected from miR-325, miR-134, miR-198, miR-721, miR-515-3p, miR-680, miR-601, miR-206, miR-202, miR-671, miR-381, miR-630, miR-759, miR-564, miR-709, miR-513, and miR-298, and any combination of two or more of the foregoing, wherein the down-regulation or under-expression of one or more of the aforementioned miRNA, when compared to a control, indicates that the subject has asthma.

In some embodiments, the present invention provides methods for determining whether a subject has asthma exacerbation, asthma by characterizing one or a combination of biomarkers selected from Tables 5-7 and FIG. 4. In some embodiments, the present invention provides methods for diagnosing asthma exacerbation by characterizing miRNA selected from miR-325, miR-134, miR-198, miR-721, miR-515-3p, miR-680, miR-601, miR-206, miR-202, miR-671, miR-381, miR-630, miR-759, miR-564, miR-709, miR-513, miR-298, and any combination thereof.

In some embodiments, the present invention provides methods for nasal polyps by characterizing one or a combination of biomarkers selected from Tables 8-10.

In one aspect, the present invention provides a method of diagnosis and/or prognosis of asthma in a young male subject. In some embodiments, the method comprises determining the level of expression of one or more microRNA biomarkers that are up-regulated or over-expressed in young male patients are shown in Tables 5 and 7 and FIGS. 3 and 5 (biomarkers with young male/old male fold change >3, 2, 1, or 0). In some embodiments, the microRNA biomarkers diagnosing whether a young male has asthma is selected from miR-15b, miR-20a, let-7d, miR-721, miR-30d, miR-320, or any combination thereof, wherein the up-regulation or over-expression of the one or more aforementioned miRNA biomarkers in a young male subject's biological sample, when compared to a control, indicates that the young male subject has asthma. In an embodiment, the young male subject is a human less than 50-year-old, or of any age younger including, but not limited to, 45-, 40-, 35-, 30-, 25-, 20-, 15-, 10, or 5-year old.

In some embodiments, the present invention provides a method of diagnosis and/or prognosis of asthma in an old male subject. In some embodiments, the method comprises determining the level of expression of one or more microRNA biomarkers microRNA biomarkers that are up-regulated or over-expressed in old male patients are shown in Tables 5 and 7 and FIGS. 3 and 5 (biomarkers with young male/old male fold change <−3, −2, −1, or 0). In some embodiments, the microRNA biomarkers diagnosing whether an old male has asthma is selected from miR-423-5p, miR-762, miR-466-5p, miR-711, or any combination thereof, wherein the up-regulation or over-expression of the one or more aforementioned miRNA biomarkers in an old male subject's biological sample, when compared to a control, indicates that the old male subject has asthma. In an embodiment, the old male subject is a human older than 50-year-old, or of any age older including, but not limited to, 55-, 60-, 65-, 70-, 75, or 80-year old.

In some embodiments, the present invention provides a method of diagnosis and/or prognosis of asthma in a young female subject. In some embodiments, the method comprises determining the level of expression of one or more microRNA biomarkers microRNA biomarkers that are up-regulated or over-expressed in young female patients are shown in Tables 6 and 7 and FIGS. 4 and 5 (biomarkers with young female/old female fold change >3, 2, 1, or 0). In some embodiments, the microRNA biomarkers diagnosing whether a young female has asthma is selected from miR-466a-5p, miR-21, miR-214, miR-575, miR-513p, let-7f, or any combination thereof, wherein the up-regulation or over-expression of the one or more aforementioned miRNA biomarkers in a young female subject's biological sample, when compared to a control, indicates that the young female subject has asthma. In an embodiment, the young female subject is a human less than 50-year-old, or of any age younger including, but not limited to, 45-, 40-, 35-, 30-, 25-, 20-, 15-, 10, or 5-year old.

In some embodiments, the present invention provides a method of diagnosis and/or prognosis of asthma in an old female subject. In some embodiments, the method comprises determining the level of expression of one or more microRNA biomarkers microRNA biomarkers that are up-regulated or over-expressed in old female patients are shown in Tables 6 and 7 and FIGS. 4 and 5 (biomarkers with young female/old female fold change <−3, −2, −1, or 0). In some embodiments, the microRNA biomarkers diagnosing whether an old female has asthma is selected from miR-15b, let-7i, miR-30d, miR-759, miR-513a-5p, miR-584, miR-637, miR-610, miR-762, or any combination thereof, wherein the up-regulation or over-expression of the one or more aforementioned miRNA biomarkers in an old female subject's biological sample, when compared to a control, indicates that the old female subject has asthma In an embodiment, the old female subject is a human older than 50-year-old, or of any age older including, but not limited to, 55-, 60-, 65-, 70-, 75, or 80-year old.

In some embodiments, the present invention provides methods for diagnosing nasal polyps by characterizing miRNA selected from miR-765, miR-705, let-7f-1, let7d, miR-494, let-7a, miR-23b, miR-191, and miR-21, or any combination thereof, wherein the up-regulation or over-expression of the one or more aforementioned miRNA biomarkers, when compared to a control, indicates that the subject has a nasal polyp.

In some embodiments, the present invention provides methods for diagnosing asthma by characterizing miRNA selected from miR-721, miR-295, miR-690, miR-302b, miR-466a-5p, miR-15a, miR-759, miR-202, miR-302, and miR-608, or any combination thereof, wherein the down-regulation or under-expression of the one or more aforementioned miRNA biomarkers, when compared to a control, indicates that the subject has a nasal polyp.

### Screening Assays for Therapeutic Agents for Airway Diseases

In one embodiment, the present invention provides a method for screening for candidate therapeutic agents for treatment of asthma comprising the steps of:

assaying miRNA expression levels in a control sample to determine baseline miRNA expression levels for the sample;

contacting a test sample with one or more therapeutic agents and one or more asthma-trigger agents;

assaying the resulting miRNA expression level in the test sample;

comparing the miRNA expression level in the test sample to the baseline miRNA expression level to determine the treated miRNA deviation;

contacting a control sample with the one or more asthma-trigger agents;

assaying the resulting miRNA expression level in the control sample to determine an exposed control;

comparing the miRNA expression level in the exposed control to the baseline miRNA expression level to determine the exposed miRNA deviation; and

comparing the treated miRNA deviation with the exposed miRNA deviation, wherein a reduced deviation in the treated miRNA deviation relative to the exposed miRNA deviation identifies a candidate asthma therapeutic agent.

In some embodiments, the miRNA is selected from one or any combination of two or more miRNA biomarkers selected from Tables 2-7 and FIGS. 1-5.

### Treatment of Airway Diseases

Another aspect of the invention provides methods for treatment of airway disease. In one embodiment, the method comprises modulating one or more miRNA that are up-regulated, down-regulated, and/or disregulated in airway diseases. In one embodiment, the subject is diagnosed with an airway disease, such as an upper airway disease.

In one embodiment, the treatment method comprises modulating one or more miRNA level up-regulated, down-regulated, and/or disregulated in airway diseases. In some embodiments, the method comprises modulating and/or inhibiting one or more miRNAs up-regulated in an airway disease, such as asthma, asthma exacerbation, nasal polyps and sinusitis. In some embodiments, the method comprises modulating and/or increasing one or more miRNAs down-regulated in an airway disease, such as asthma, asthma exacerbation, nasal polyps and sinusitis. FIGS. 1-7 and Tables 2-10 provide embodiments of miRNA up-regulated, down-regulated, and/or disregulated in asthma, asthma exacerbation, nasal polyps and sinusitis.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression and/or severity of an undesired physiological change or a diseased condition.

In one embodiment, the treatment method comprises administering to a subject who has an airway disease, one or more miRNA listed in Tables 2-10 and FIGS. 1-7 that are down-regulated in said airway disease.

The term “effective amount,” as used herein, refers to an amount that is capable of treating or ameliorating a disease or condition or otherwise capable of producing an intended therapeutic effect.

Another aspect of the subject invention pertains to the use of miRNA biomarkers of the present invention as targets for screening for therapeutics for airway disease. The therapeutic agent can be a drug, chemical, compound, protein or peptide, or a nucleic acid molecule (e.g., DNA, RNA such as siRNA).

### Therapeutic Compositions and Routes of Administration

The subject invention further provides therapeutic compositions that contain a therapeutically effective amount of the therapeutic agent of the subject invention and a pharmaceutically acceptable carrier or adjuvant.

The therapeutic agent used in the therapies can be in a variety of forms. These include for example, solid, semi-solid, and liquid dosage forms, such as tablets, pills, powders, liquid solutions or suspensions, suppositories, and injectable and infusible solutions. The preferred form depends on the intended mode of administration and therapeutic application.

In a preferred embodiment, the composition is formulated in accordance with routine procedures as a pharmaceutical composition adapted for local injection administration to human beings. Typically, compositions for local injection administration are solutions in sterile isotonic aqueous buffer. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The subject invention also provides for a therapeutic method by administering therapeutic or pharmaceutical compositions in a form that can be combined with a pharmaceutically acceptable carrier. In this context, the compound may be, for example, isolated or substantially pure. The term “carrier” refers to a diluent, adjuvant, excipient, or vehicle with which the compound is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin.

Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents.

The amount of active ingredient that may be combined with the carrier materials to produce a single dosage form will vary, depending such as the type of the condition and the subject to be treated. The amount of active ingredient that may be combined with the carrier materials to produce a single dosage form will vary, depending such as the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The compound of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

The therapeutic or pharmaceutical compositions of the subject invention can also be formulated as neutral or salt forms. Pharmaceutically acceptable salts include salts derived from hydrochloric, phosphoric, acetic, oxalic, or tartaric acids, etc., and salts derived from sodium, potassium, ammonium, calcium, ferric hydroxides, isopropylamine, triethylamine, 2-ethylamino ethanol, histidine, procaine, etc.

The compositions of the subject invention can be administered to the subject being treated by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a subject.

The amount of the therapeutic or pharmaceutical composition of the subject invention effective in the treatment of an airway disease will depend on a variety of factors, such as the route of administration and the seriousness of the condition, and should be decided according to the judgment of the practitioner and each patient's circumstances. In general, the dosage ranges from about 0.01 μg/kg to about 10 mg/kg, about 0.01 μg/kg to about 1 mg/kg, about 0.01 μg/kg to about 100 μg/kg, about 0.01 μg/kg to about 10 μg/kg, or about 0.01 μg/kg to about 1 μg/kg. Such a unit dose may be administered once to several times (e.g. two, three and four times) every two weeks, every week, or every day.

In one embodiment, the compounds and compositions of the subject invention and any second therapeutic agent are administered simultaneously or sequentially to the patient, with the second therapeutic agent being administered before, after, or both before and after treatment with the compounds of the subject invention. Sequential administration may involve treatment with the second therapeutic agent on the same day (within 24 hours) of treatment with the subject compound. Sequential administration may also involve continued treatment with the second therapeutic agent on days that the subject compound is not administered.

In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

### Sample

The present invention provides a method of diagnosis, prognosis, and treatment of airway diseases such as asthma, asthma exacerbation, nasal polyps and sinusitis on at least one sample obtained from an individual. The individual may be any mammal, but is preferably a human.

The present invention concerns airway diseases such as asthma, asthma exacerbation, nasal polyps and sinusitis, and may involve obtaining more than one sample, such as two samples, such as three samples, four samples or more from individuals, and preferably the same individual, is of importance. This allows the relative comparison of expression both as in the presence or absence of at least one nucleic acid and/or the level of expression of the at least one nucleic acid between the two samples. Alternatively, a single sample may be compared against a “standardized” sample, such a sample comprising material or data from several samples, preferably also from several individuals.

### Sample Preparation

Before analyzing the sample, it will often be desirable to perform one or more sample preparation operations upon the sample. Typically, these sample preparation operations will include such manipulations as concentration, suspension, extraction of intracellular material, e.g., nucleic acids from tissue/whole cell samples and the like, amplification of nucleic acids, fragmentation, transcription, labelling and/or extension reactions.

Nucleic acids, especially RNA and specifically miRNA can be isolated using any techniques known in the art. There are two main methods for isolating RNA: phenol-based extraction and silica matrix or glass fiber filter (GFF)-based binding. Phenol-based reagents contain a combination of denaturants and RNase inhibitors for cell and tissue disruption and subsequent separation of RNA from contaminants. Phenol-based isolation procedures can recover RNA species in the 10-200-nucleotide range e.g., miRNAs, 5S rRNA, 5.8S rRNA, and U1 snRNA. If a sample of “total” RNA was purified by the popular silica matrix column or GFF procedure, it may be depleted in small RNAs. Extraction procedures such as those using Trizol or TriReagent, however will purify all RNAs, large and small, and are the recommended methods for isolating total RNA from biological samples that will contain miRNAs/siRNAs.

Any method required for the processing of a sample prior to detection by any of the methods noted herein falls within the scope of the present invention. These methods are typically well known by a person skilled in the art.

### Detection

It is within the general scope of the present invention to provide methods for the detection of miRNA. An aspect of the present invention relates to the detection of the miRNA sequences as described in the plots and graphs of the figures contained herein. By detection is meant both 1) detection in the sense of presence versus absence of one or more miRNAs as well as 2) the registration of the level or degree of expression of one or more miRNAs, depending on the method of detection employed.

The detection of one or more nucleic acid molecules allows for the classification, diagnosis and prognosis of an airway disease. The classification of a disease is of relevance both medically and scientifically and may provide important information useful for the diagnosis, prognosis and treatment of the disease. The diagnosis of a disease is the affirmation of the presence of the disease based, as is the object of the present invention, on the expression of at least one miRNA herein also referred to as a nucleic acid molecule. Prognosis is the estimate or prediction of the probable outcome of a disease and the prognosis of a disease is greatly facilitated by increasing the amount of information on the particular disease. The method of detection is thus a central aspect of the present invention.

Any method of detection falls within the general scope of the present invention. The detection methods may be generic for the detection of nucleic acids especially RNA, or be optimized for the detection of small RNA species, as both mature and precursor miRNAs fall into this category or be specially designed for the detection of miRNA species. The detection methods may be directed towards the scoring of a presence or absence of one or more nucleic acid molecules or may be useful in the detection of expression levels.

The detection methods can be divided into two categories herein referred to as in situ methods or screening methods. The term in situ method refers to the detection of nucleic acid molecules in a sample wherein the structure of the sample has been preserved. This may thus be a biopsy wherein the structure of the tissue is preserved. In situ methods are generally histological i.e. microscopic in nature and include but are not limited to methods such as: in situ hybridization techniques and in situ PCR methods.

Screening methods generally employ techniques of molecular biology and most often require the preparation of the sample material in order to access the nucleic acid molecules to be detected. Screening methods include, but are not limited to methods such as: Array systems, affinity matrices, Northern blotting and PCR techniques, such as real-time quantitative RT-PCR.

### Probe

It is an object of the present invention to provide a probe which can be used for the detection of a nucleic acid molecule as defined herein. A probe as defined herein is a specific sequence of a nucleic acid used to detect nucleic acids by hybridization. A nucleic acid is also here any nucleic acid, natural or synthetic such as DNA, RNA, LNA or PNA. A probe may be labeled, tagged or immobilized or otherwise modified according to the requirements of the detection method chosen. A label or a tag is an entity making it possible to identify a compound to which it is associated. It is within the scope of the present invention to employ probes that are labeled or tagged by any means known in the art such as but not limited to: radioactive labeling, fluorescent labeling and enzymatic labeling. Furthermore the probe, labeled or not, may be immobilized to facilitate detection according to the detection method of choice and this may be accomplished according to the preferred method of the particular detection method.

### Detection Methods

An aspect of the present invention regards the detection of nucleic acid molecules by any method known in the art. In the following are given examples of various detection methods that can be employed for this purpose, and the present invention includes all the mentioned methods, but is not limited to any of these.

### In Situ Hybridization

In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules as herein described. Sample cells are treated to increase their permeability to allow the probe to enter the cells, the probe is added to the treated cells, allowed to hybridize at pertinent temperature, and then excess probe is washed away. A complementary probe is labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the miRNAs mentioned herein.

An aspect of the present invention includes the method of detection by in situ hybridization as described herein.

### In Situ PCR

In situ PCR is the PCR based amplification of the target nucleic acid sequences prior to ISH. For detection of RNA, an intracellular reverse transcription (RT) step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR the cells are cytocentrifugated onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens. Detection of intracellular PCR-products is achieved by one of two entirely different techniques. In indirect in situ PCR by ISH with PCR-product specific probes, or in direct in situ PCR without ISH through direct detection of labelld nucleotides (e.g. digoxigenin-11-dUTP, fluorescein-dUTP, 3H-CTP or biotin-16-dUTP) which have been incorporated into the PCR products during thermal cycling.

An embodiment of the present invention concerns the method of in situ PCR as mentioned herein above for the detection of nucleic acid molecules as detailed herein.

### Microarray

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used e.g. to measure the expression levels of large numbers of mRNAs/miRNAs simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

An aspect of the present invention regards the use of microarrays for the expression profiling of miRNAs in airway disease. For this purpose, and by way of example, RNA is extracted from a cell or tissue sample, the small RNAs (18-26-nucleotide RNAs) are size-selected from total RNA using denaturing polyacrylamide gel electrophoresis (PAGE). Then oligonucleotide linkers are attached to the 5′ and 3′ ends of the small RNAs and the resulting ligation products are used as templates for an RT-PCR reaction with 10 cycles of amplification. The sense strand PCR primer has a Cy3 fluorophore attached to its 5′ end, thereby fluorescently labelling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA.

Several types of microarrays can be employed such as spotted oligonucleotide microarrays, pre-fabricated oligonucleotide microarrays or spotted long oligonucleotide arrays.

In spotted oligonucleotide microarrays the capture probes are oligonucleotides complementary to miRNA sequences. This type of array is typically hybridized with amplified

PCR products of size-selected small RNAs from two samples to be compared that are labelled with two different fluorophores. Alternatively, total RNA containing the small RNA fraction (including the miRNAs) is extracted from the abovementioned two samples and used directly without size-selection of small RNAs, and 3′ end labeled using T4 RNA ligase and short RNA linkers labelled with two different fluorophores. The samples can be mixed and hybridized to one single microarray that is then scanned, allowing the visualization of up-regulated and down-regulated miRNA genes in one go. The downside of this is that the absolute levels of gene expression cannot be observed, but the cost of the experiment is reduced by half. Alternatively, a universal reference can be used, comprising of a large set of fluorophore-labelled oligonucleotides, complementary to the array capture probes.

In pre-fabricated oligonucleotide microarrays or single-channel microarrays, the probes are designed to match the sequences of known or predicted miRNAs. There are commercially available designs that cover complete genomes from companies such as Affymetrix, or Agilent. These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing. More recently, Maskless Array Synthesis from NimbleGen Systems has combined flexibility with large numbers of probes. Arrays can contain up to 390,000 spots, from a custom array design.

An embodiment of the present invention concerns the method of microarray use and analysis as described herein.

### PCR

The terms “PCR reaction”, “PCR amplification”, “PCR”, “pre-PCR”, “Q-PCR”, “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence based amplification and Q Beta Replicase systems. The products formed by said amplification reaction may or may not be monitored in real time or only after the reaction as an end-point measurement.

### Real-Time Quantitative RT-PCR

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This is commonly used for the purpose of determining whether a genetic sequence is present or not, and if it is present the number of copies in the sample. There are 3 methods which vary in difficulty and detail. Like other forms of polymerase chain reaction, the process is used to amplify DNA samples, using thermal cycling and a thermostable DNA polymerase.

The three commonly used methods of quantitative polymerase chain reaction are through agarose gel electrophoresis, the use of SYBR Green, a double stranded DNA dye, and the fluorescent reporter probe. The latter two of these three can be analysed in real-time, constituting real-time polymerase chain reaction method.

Agarose gel electrophoresis is the simplest method, but also often slow and less accurate then other methods, depending on the running of an agarose gel via electrophoresis. It cannot give results in real time. The unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown. This method is generally used as a simple measure of whether the probe target sequences are present or not, and rarely as ‘true’ Q-PCR.

Using SYBR Green dye is more accurate than the gel method, and gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artefacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescent reporter probe method is the most accurate and most reliable of the methods. It uses a sequence-specific nucleic acid based probe so as to only quantify the probe sequence and not all double stranded DNA. It is commonly carried out with DNA based probes with a fluorescent reporter and a quencher held in adjacent positions, so-called dual-labelled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerisation continues it reaches the probe bound to its complementary sequence, which is then hydrolysed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolysed dual-labelled probe in each PCR cycle is monitored, which allows accurate determination of the final, and so initial, quantities of DNA.

Any method of PCR that can determine the expression of a nucleic acid molecule as defined herein falls within the scope of the present invention. A preferred embodiment of the present invention includes the real-time quantitative RT-PCR method, based on the use of either SYBR Green dye or a dual-labelled probe for the detection and quantification of nucleic acids according to the herein described.

### Northern Blot Analysis

An aspect of the present invention includes the detection of the nucleic acid molecules herein disclosed by techniques such as Northern blot analysis. Many variations of the protocol exist.

### EXEMPLIFIED EMBODIMENTS

### Embodiment 1

A method for preparing a microRNA (miRNA) expression profile for an airway disease, comprising: determining the level of expression of an miRNA in a sample, thereby preparing the miRNA expression profile.

### Embodiment 2

The method of embodiment 1, wherein the miRNA comprises one or more miRNAs of Tables 1-10 and FIGS. 1-7.

### Embodiment 3

The method of embodiment 1 or 2, wherein the upper airway disease comprises asthma.

### Embodiment 4

The method of embodiment 1 or 2, wherein the airway disease comprises asthma exacerbation.

### Embodiment 5

The method of embodiment 1 or 2, wherein the upper airway disease comprises nasal polyps.

### Embodiment 6

The method of embodiment 3, wherein the miRNA comprises one or more miRNAs of Tables 2-4 and FIGS. 1-4.

### Embodiment 7

The method of embodiment 3, wherein the miRNA comprises one or more miRNAs up-regulated in asthma shown in Tables 2-4 and FIGS. 1-4.

### Embodiment 8

The method of embodiment 7, wherein the miRNA profile comprises or consists of the expression level of one or more miRNAs selected from among miR-705, miR-575, let-7d, miR-173p, miR-423-5p, miR-611, miR-674, let-7f-1, miR-23b, miR-223, miR-142-3p, let-7c, miR-25, miR-15b, let-7g, and miR-542-5p, and miR-370, and wherein up-regulation (increase) of the one or more miRNAs compared to a normal control is indicative of asthma.

### Embodiment 9

The method of embodiment 3, wherein the miRNA comprises one or more miRNAs down-regulated in asthma shown in Tables 2-4 and FIGS. 1-4.

### Embodiment 10

The method of embodiment 9, wherein the miRNA profile comprises or consists of the expression level of one or more miRNAs selected from among miR-325, mmu-miR-134, miR-198, miR-721, miR-515-3p, miR-680, miR-601, mmu-miR-206, miR-202, miR-671, mmu-miR-381, hsa-miR-630, miR-759, miR-564, miR-709, miR-513, and mmu-miR-298, and wherein down-regulation (decrease) of the one or more miRNAs compared to a normal control is indicative of asthma.

### Embodiment 11

The method of embodiment 4, wherein the miRNA comprises one or more miRNAs of Tables 5-7.

### Embodiment 12

The method of embodiment 4, wherein the miRNA comprises one or more miRNAs up-regulated in in asthma exacerbation shown in Tables 5-7.

### Embodiment 13

The method of embodiment 5, wherein the miRNA comprises one or more miRNAs up-regulated in nasal polyp shown in Tables 8-10 and FIGS. 6-7.

### Embodiment 14

The method of embodiment 17, wherein the miRNA profile comprises or consists of the expression level of one or more miRNAs selected from among miR-765, miR-705, let-7f-1, let7d, miR-494, let-7a, miR-23b, miR-191, and miR-21, and wherein up-regulation (increase) of the one or more miRNAs compared to a normal control is indicative of nasal polyps.

### Embodiment 15

The method of embodiment 5, wherein the miRNA comprises one or more miRNAs down-regulated in nasal polyp shown in Tables 8-10 and FIGS. 6-7.

### Embodiment 16

wherein the miRNA profile comprises or consists of the expression level of one or more miRNA selected from among miR-721, miR-295, miR-690, miR-302b, miR-466a-5p, miR-15a, miR-759, miR-202, miR-302, and miR-608, and wherein down-regulation (decrease) of the one or more miRNAs compared to a normal control is indicative of nasal polyps.

### Embodiment 17

The method of any one of embodiments 1-16, wherein the sample is a blood sample.

### Embodiment 18

A method for detecting an airway disease in a subject, comprising:

a) determining a miRNA profile in a biological sample from the subject; and

b) characterizing the miRNA profile level, wherein the miRNA of the profile is selected from one or more miRNA of Tables 1-10 and FIGS. 1-7.

### Embodiment 19

The method of embodiment 22, wherein the airway disease is asthma, and wherein the miRNA comprises one or more miRNAs of Tables 2-4 and FIGS. 1-4.

### Embodiment 20

The method of embodiment 19, wherein the airway disease is nasal polyps, and wherein the miRNA comprises one or more miRNAs of Tables 8-10 and FIGS. 6-7.

### Embodiment 21

A method for treating asthma, comprising:

(a) modulating and/or inhibiting one or more miRNA of one or more miRNAs up-regulated in asthma shown in Tables 2-4 and FIGS. 1-4; and/or

(b) increasing level of one or more miRNAs down-regulated in asthma shown in Tables 2-4 and FIGS. 1-4.

### Embodiment 22

A method for treating asthma exacerbation, comprising:

(a) modulating and/or inhibiting one or more miRNA of one or more miRNAs up-regulated in asthma exacerbation shown in Tables 2-4 and FIGS. 1-4; and/or

(b) increasing level of one or more miRNAs down-regulated in asthma exacerbation shown in Tables 2-4 and FIGS. 1-4.

### Embodiment 23

A method for treating nasal polyps, comprising:

(a) modulating and/or inhibiting one or more miRNA of one or more miRNAs up-regulated in nasal polyps shown in Tables 2-4 and FIGS. 1-4; and/or

(b) increasing level of one or more miRNAs down-regulated in asthma excerbation shown in Tables 5-7.

### Embodiment 24

A method for diagnosing an airway disease in a subject, comprising:

a) determining an miRNA profile in a biological sample from the subject; and

b) characterizing the subject's miRNA profile level, wherein the miRNA is selected from one or more miRNA of Tables 1-10 and FIGS. 1-7.

### Embodiment 25

The method of embodiment 24, wherein the airway disease is asthma, and wherein the miRNA comprises one or more miRNAs of Tables 2-4 and FIGS. 1-4 (such as one or more miRNAs from embodiment 8 or embodiment 10).

### Embodiment 26

The method of embodiment 24, wherein the airway disease is nasal polyps, and wherein the miRNA comprises one or more miRNAs of Tables 8-10 and FIGS. 6-7 (such as one or more miRNAs from embodiment 18 or embodiment 20).

### Embodiment 27

A method for predicting a subject's risk of asthma exacerbation, comprising:

a) determining an miRNA profile in a biological sample from the subject; and

b) characterizing the subject's miRNA profile level, wherein the miRNA is selected from one or more miRNA of Tables 5-7 (such as one or more miRNAs from embodiment 13 or embodiment 15).

### Embodiment 28

A probe array for performing the method of any preceding embodiment, comprising a plurality of probes that hybridizes to one or more miRNAs of Tables 1-10 and FIGS. 1-7.

### Embodiment 29

The probe array of embodiment 28, further comprising a solid support with the plurality of probes attached thereto.

### Embodiment 30

A kit for performing the method of any preceding embodiment, comprising the probe array of embodiment 22 or 23 and instructions for carrying out the method.

### Embodiment 31

An isolated precursor miRNA that increases the level or activity of one or more target miRNAs of Tables 1-10 and FIGS. 1-7, wherein a decreased target miRNA level relative to normal is indicative of an airway disease or increased risk of exacerbation of an airway disease.

### Embodiment 32

A method for treating an airway disease in a subject selected from among asthma, asthma exacerbation, and/or nasal polyps, comprising:

a) determining a miRNA profile in a biological sample from the subject; b) characterizing the miRNA profile level, wherein the miRNA of the profile is selected from one or more miRNA of Tables 1-10 and FIGS. 1-7; and

c) treating the subject for the airway disease if the miRNA profile is one which indicates the presence of asthma, asthma exacerbation (or risk thereof), and/or nasal polyps.

### Embodiment 33

The method of embodiment 32, further comprising obtaining the sample from the subject prior to determining the miRNA profile in the biological sample.

### Embodiment 34

The method of embodiment 32, wherein the airway disease comprises asthma, and wherein the miRNA comprises one or more miRNAs of Tables 2-4 and FIGS. 1-4.

### Embodiment 35

The method of embodiment 34, wherein the miRNA profile comprises or consists of the expression level of one or more miRNAs selected from among miR-705, miR-575, let-7d, miR-173p, miR-423-5p, miR-611, miR-674, let-7f-1, miR-23b, miR-223, miR-142-3p, let-7c, miR-25, miR-15b, let-7g, and miR-542-5p, and miR-370, and wherein up-regulation (increase) of the one or more miRNAs compared to a normal control is indicative of asthma.

### Embodiment 36

The method of embodiment 34, wherein the miRNA profile comprises or consists of the expression level of one or more miRNAs selected from among miR-325, mmu-miR-134, miR-198, miR-721, miR-515-3p, miR-680, miR-601, mmu-miR-206, miR-202, miR-671, mmu-miR-381, hsa-miR-630, miR-759, miR-564, miR-709, miR-513, and mmu-miR-298, and wherein down-regulation (decrease) of the one or more miRNAs compared to a normal control is indicative of asthma.

### Embodiment 37

The method of embodiment 34, wherein the airway disease comprises nasal polyps, and wherein the miRNA comprises one or more miRNAs of Tables 8-10 and FIGS. 6-7.

### Embodiment 38

The method of embodiment 37, wherein the miRNA profile comprises or consists of the expression level of one or more miRNAs selected from among miR-765, miR-705, let-7f-1, let7d, miR-494, let-7a, miR-23b, miR-191, and miR-21, and wherein up-regulation (increase) of the one or more miRNAs compared to a normal control is indicative of nasal polyps.

### Embodiment 39

The method of embodiment 37, wherein the miRNA profile comprises or consists of the expression level of one or more miRNA selected from among miR-721, miR-295, miR-690, miR-302b, miR-466a-5p, miR-15a, miR-759, miR-202, miR-302, and miR-608, and wherein down-regulation (decrease) of the one or more miRNAs compared to a normal control is indicative of nasal polyps.

### Embodiment 40

The method of embodiment 32, wherein the airway disease comprises asthma exacerbation, and wherein the miRNA is selected from one or more miRNA of Tables 5-7.

### Materials and Methods

Detection and Quantification of miRNA

miRNAs are detected and quantified using the novel RT-qPCR miRNA Taqman assay (UQmiR) and a computer program (QmiR) developed by the inventors. This miRNA profiling assay requires only one RT reaction and one hydrolysis probe for detection of all miRNAs. The miRNA assay uses one universal RT primer, a common reverse primer, and individual miRNA-specific forward primers. The inventors designed (using the QmiR program) and synthesized 1500 miRNA primers that cover all known human and mouse miRNA genes. These primers have ±1° C. of Tm variations. UQmiR is at least two times more sensitive than the commercially available Taqman assay and specifically detect mature miRNAs but not other non-miRNA sequences. This miRNA assay can sensitively and specifically detect 88 out of 94 miRNAs in just 0.8 μl of plasma for each miRNA.

Plasma miRNA Extraction

Equal volumes (100 μl) of 80 plasma samples from each of the five groups are pooled and plasma miRNAs are isolated using the ultracentrifugation procedure as previously described (P. Fernandez-Llama et al., Kidney Int 77, 736 (April, 2010). RNA quality is determined by Experion automated electrophoresis system (Bio-Rad).

### RT-qPCR

The UQmiR assay for profiling plasma miRNAs is conducted in triplicates following standard RT qPCR procedures as described (E. M. Kroh, R. K. Parkin, P. S. Mitchell, M. Tewari, Methods 50, 298, 2010) with some modifications, using 384 well plates and the ABI PRISM 7900HT thermocycler. miRNA levels are normalized by the mean value method (P. Mestdagh et al., Genome Biol 10, R64, 2009). miRNAs showing significant differences in expression levels (2 fold and greater) between each group are confirmed by mirVana™ miRNA Detection Kit (Ambion), which can detect as little as 10 attomoles of target RNA.

### Data Analysis and Quality Control

To obtain valid data, the guidelines of the minimum information for publication of quantitative real-time PCR experiments (MIQE) (8) and the statistical methods described previously (S. A. Bustin et al., Clin Chem 55, 611, 2009) (9) are followed.

### Alternative Methods

Additional plasma samples can be obtained locally from the Division of Allergy and Immunology at the University of South Florida, College of Medicine. Ultrafiltration or antibody precipitation methods for plasma miRNA extraction can be used to obtain high quality plasma miRNAs. To quantitate absolute miRNA levels in the plasma, absolute qPCR can be carried out using synthetic miRNAs spiked into plasma samples. Dot-blot miRNA array (11), standard SYBR Green or Taqman qPCR assay can be carried out to confirm the results obtained by the novel UQmiR array.

### EXAMPLES

Following are examples which illustrate procedures for practicing the invention. These examples should not be construed as limiting. All percentages are by weight and all solvent mixture proportions are by volume unless otherwise noted.

### Example 1

### Detection of microRNA Biomarkers of Inflammation in Healthy Human Plasma Samples

The RT-qPCR miRNA Taqman assay (UQmiR) and the computer program (QmiR) developed by the inventors sensitively and specifically detect 88 out of 94 miRNAs in just 0.8 μl of plasma sample for each miRNA. The relative expression levels were normalized to mean average. 94 miRNAs having the highest expression levels among about 800 miRNAs in mouse spleens, as determined by miRNA dot blot array, were selected for qPCR miRNA array assay.

Table 1 shows relative plasma miRNA expression levels of human plasma miRNAs pooled from five normal volunteers. The miRNA levels are measured by the RT-qPCR miRNA Taqman assay developed by the present inventors. Most of the 94 miRNAs that have the highest expression levels in mouse spleens are also detectable in human plasma (Table 1). miRNAs with high expression levels are: miR-680, miR-27a, miR-134, miR-206, miR-493, Let-7i, miR-92, miR-422b, miR-540, miR-422b, miR-564, miR-770-3p, miR-142-3p and miR-150 (Table 1). Positive numbers denote expression levels above average, while negative numbers denote expression levels below average. “-” denotes undetected.

### Example 2

### Biomarkers of Asthma

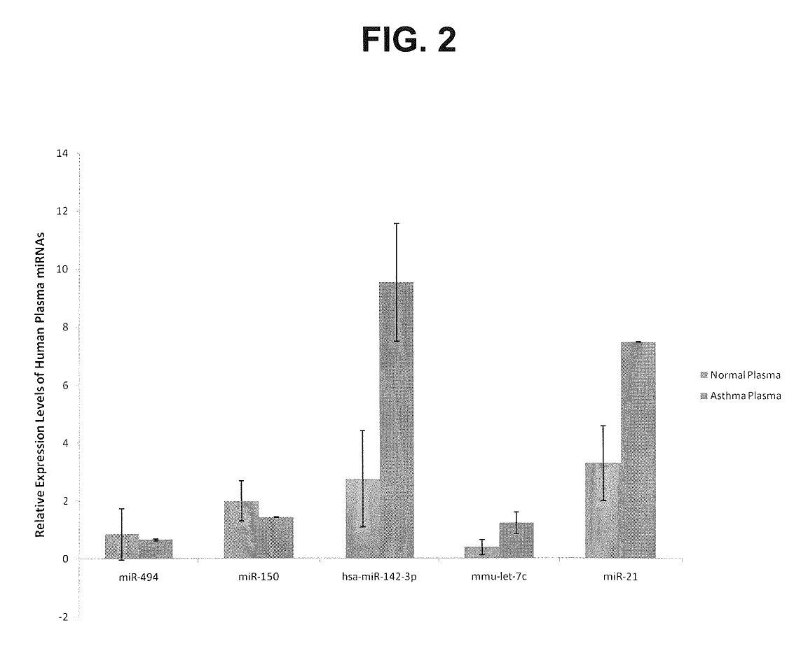

Plasma miRNA expression levels are different between asthma patients and normal volunteers (Table 2, FIG. 1 and FIG. 2). miRNAs up-regulated in asthma patients, when compared to normal controls, include miR-705, miR-575, let-7d, miR-173p, miR-423-5p, miR-611, miR-674, let-7f-1, miR-23b, miR-223, miR-142-3p, let-7c, miR-25, miR-15b, let-7g, and miR-542-5p, miR-370. Table 2 shows fold change of plasma miRNA expression levels in asthma patients, when compared to normal volunteers. Positive numbers denote that the miRNA expression is up-regulated in asthma, while negative numbers denote that the miRNA expression is down-regulated in asthma. “-” denotes undetected. The enriched pathways targeted by the miRNAs up-regulated in asthma patients include MAPK signaling pathway, TGF-beta signaling pathway, and ErbB signaling pathway (Table 3).

Among these miRNAs, miR-142-3p, Let-7c and miR-21 are also differentially expressed in old and young mice. Those miRNA target enriched pathways, including MAPK signaling pathway, TGF-beta signaling pathway and ErbB signaling pathway (Table 3).

The miRNAs down-regulated in asthma patients, when compared to normal controls, include miR-325, mmu-miR-134, miR-198, miR-721, miR-515-3p, miR-680, miR-601, mmu-miR-206, miR-202, miR-671, mmu-miR-381, hsa-miR-630, miR-759, miR-564, miR-709, miR-513, and mmu-miR-298. Those enriched pathways targeted by the miRNAs down-regulated in asthma patients include TGF-beta signaling pathway, MAPK signaling pathway, mTOR signaling pathway, and Wnt signaling pathway (Table 4).

### Example 3

### Biomarkers of Asthma Exacerbation

Table 5 lists the fold-change of plasma miRNA expression levels for male asthma patients with exacerbation. Relative plasma miRNA expression levels of human plasma miRNAs pooled from 7 young male patients (23 to 48 years old) and 5 old male patients (56 to 71 years old) are measured by measured by the RT-qPCR miRNA Taqman assay. Table 6 lists the fold-change of plasma miRNA expression levels for female asthma patients with exacerbation. Relative plasma miRNA expression levels of human plasma miRNAs pooled from 11 young female patients (25 to 47 years old) and 6 old female patients (58 to 66 years old) are measured the by RT-qPCR miRNA Taqman assay. Table 7 compares miRNA expression levels in four groups of asthma patients with exacerbation.

### Example 4

### Biomarkers of Patients with Nasal Polyps

FIG. 6 identifies biomarkers of patients with nasal polyps. The miRNA biomarkers up-regulated in patients with nasal polyps, when compared to control healthy controls, include miR-765, miR-705, let-7f-1, let7d, miR-494, let-7a, miR-23b, miR-191, and miR-21. miRNA biomarkers down-regulated in patients with nasal polyps, when compared to control healthy controls, include miR-721, miR-295, miR-690, miR-302b, miR-466a-5p, miR-15a, miR-759, miR-202, miR-302, and miR-608.

Table 8 shows fold change of miRNA expression levels in nasal polyps, when compared to normal nasal epithelial cells. Relative plasma miRNA expression levels human plasma miRNAs pooled from 6 patients and 5 normal volunteers are measured by the RT-qPCR miRNA Taqman assay.

miRNA biomarkers up-regulated in nasal polyps include miR-765, miR-705, let-7f-1, let7d, miR-494, let-7a, miR-23b, miR-191, and miR-21. miRNA biomarkers down-regulated in nasal polyps include miR-721, miR-295, miR-690, miR-302b, miR-466a-5p, miR-15a, miR-759, miR-202, miR-302, and miR-608.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein, supra or infra, are incorporated by reference in their entirety, including all figures and tables, to the extent they are not inconsistent with the explicit teachings of this specification

### REFERENCES

S. M. Ho, J Allergy Clin Immunol 126, 453 (September, 2010).
A. Zomer et al., Commun Integr Biol 3, 447 (September, 2010).
P. Fernandez-Llama et al., Kidney Int 77, 736 (April, 2010).
E. M. Kroh, R. K. Parkin, P. S. Mitchell, M. Tewari, Methods 50, 298 (April, 2010).
P. Mestdagh et al., Genome Biol 10, R64 (2009).
J. S. Yuan, A. Reed, F. Chen, C. N. Stewart, Jr., BMC Bioinformatics 7, 85 (2006). S. A. Bustin et al., Clin Chem 55, 611 (April, 2009).
J. W. Wang, J. Q. Cheng, Methods Mol Biol 414, 183 (2008).

We claim:

1. A method of detecting asthma in a subject, wherein the method comprises:
a) obtaining a biological sample from a subject;
b) determining the expression level of one or more miRNA biomarkers in the biological sample from the subject, wherein the miRNA biomarker is selected from the group consisting of miR-705, miR-575, let-7d, miR-173p, miR-423-5p, miR-611, miR-674, let-7f-1, miR-23b, miR-223, miR-142-3p, let-7c, miR-25, miR-15b, let-7g, miR-542-5p, and miR-370; and
c) comparing the expression level of the subject's miRNA biomarkers with that of a control;
wherein asthma is detected if said one or more miRNA biomarkers selected from the group consisting of miR-705, miR-575, let-7d, miR-173p, miR-423-5p, miR-611, miR-674, let-7f-1, miR-23b, miR-223, miR-142-3p, let-7c, miR-25, miR-15b, let-7g, miR-542-5p, and miR-370 are over-expressed in the subject's biological sample, when compared to a control.

2. The method of claim 1, wherein the subject is a human.

3. The method of claim 1, wherein the biological sample is a plasma sample.

4. The method of claim 1, further comprising:
determining the expression level of one or more miRNA biomarkers in the biological sample from the subject, wherein the miRNA biomarker is selected from the group consisting of miR-325, miR-134, miR-198, miR-721, miR-515-3p, miR-680, miR-601, miR-206, miR-202, miR-671, miR-381, miR-630, miR-759, miR-564, miR-709, miR-513, and miR-298, and
wherein asthma is detected if said one or more miRNA biomarkers selected from miR-325, miR-134, miR-198, miR-721, miR-515-3p, miR-680, miR-601, miR-206, miR-202, miR-671, miR-381, miR-630, miR-759, miR-564, miR-709, miR-513, and miR-298 are under-expressed in the subject's biological sample, when compared to a control.

5. The method of claim 2, wherein the subject is a male of less than 40 years of age or younger, and wherein the method comprises:
determining the expression level of one or more miRNA biomarkers in the biological sample from the subject, wherein the miRNA biomarker is selected from the group consisting of miR-15b, miR-20a, miR-721, miR-30d, miR-320, or a combination thereof; and
comparing the expression level of the subject's miRNA biomarkers with that of a control;
wherein asthma is detected if said one or more miRNA biomarkers selected from the group consisting of miR-15b, miR-20a, miR-721, miR-30d, miR-320, or a combination are over-expressed in the subject's biological sample, when compared to a control.

6. The method of claim 2, wherein the subject is a male of older than 40, wherein the method comprises:
determining the expression level of one or more miRNA biomarkers in the biological sample from the subject, wherein the miRNA biomarker is selected from the group consisting of miR-15b, miR-20a, miR-721, miR-30d, miR-320, or a combination thereof; and
comparing the expression level of the subject's miRNA biomarkers with that of a control;
wherein asthma is detected if said one or more miRNA biomarkers selected from the group consisting of miR-15b, miR-20a, miR-721, miR-30d, miR-320, or a combination are over-expressed in the subject's biological sample, when compared to a control.

7. The method of claim 2, wherein the subject is a female of 40 years of age or younger, wherein the method comprises:
determining the expression level of one or more miRNA biomarkers in the biological sample from the subject, wherein the miRNA biomarker is selected from the group consisting of miR-466a-5p, miR-21, miR-214, miR-575, and miR-513p; and
comparing the expression level of the subject's miRNA biomarkers with that of a control;
wherein asthma is detected if said one or more miRNA biomarkers selected from the group consisting of miR-466a-5p, miR-21, miR-214, miR-575, and miR-513p, or a combination are over-expressed in the subject's biological sample, when compared to a control.

8. The method of claim 2, wherein the subject is a female of older than 40, and wherein the method comprises:
determining the expression level of one or more miRNA biomarkers in the biological sample from the subject, wherein the miRNA biomarker is selected from the group consisting of miR-15b, let-7i, miR-30d, miR-759, miR-513a-5p, miR-584, miR-637, miR-610, and miR-762; and
comparing the expression level of the subject's miRNA biomarkers with that of a control;
wherein asthma is detected if said one or more miRNA biomarkers selected from the group consisting of miR-15b, let-7i, miR-30d, miR-759, miR-513a-5p, miR-584, miR-637, miR-610, and miR-762, or a combination are over-expressed in the subject's biological sample, when compared to a control.

9. A method of detecting nasal polyps in a subject, wherein the method comprises:
a) obtaining a biological sample from a subject;
b) determining the expression level of one or more miRNA biomarkers in the biological sample from the subject, wherein the miRNA biomarker is selected from the group consisting of miR-765, miR-705, let-7f-1, let7d, miR-494, let-7a, miR-23b, miR-191, and miR-21; and
c) comparing the expression level of the subject's miRNA biomarkers with that of a control;
wherein nasal polyps are detected if said one or more miRNA biomarkers selected from the group consisting of miR-765, miR-705, let-7f-1, let7d, miR-494, let-7a, miR-23b, miR-191, and miR-21 are over-expressed in the subject's biological sample, when compared to a normal control.

10. The method of claim 9, wherein the subject is a human.

11. The method of claim 9, wherein the biological sample is a nasal tissue sample.

12. The method of claim 9, further comprising:
determining the expression level of one or more miRNA biomarkers in the biological sample from the subject, wherein the miRNA biomarker is selected from the group consisting of miR-721, miR-295, miR-690, miR-302b, miR-466a-5p, miR-15a, miR-759, miR-202, miR-302, and miR-608, and
wherein nasal polyps are detected if said one or more miRNA biomarkers selected from among miR-721, miR-295, miR-690, miR-302b, miR-466a-5p, miR-15a, miR-759, miR-202, miR-302, and miR-608 are under-expressed in the subject's biological sample, when compared to a control.

13. A composition of matter comprising:
(a) probe array for determining an miRNA expression level in a sample, comprising a plurality of probes that hybridizes to one or more miRNAs miR-705, miR-575, let-7d, miR-173p, miR-423-5p, miR-611, miR-674, let-7f-1, miR-23b, miR-223, miR-142-3p, let-7c, miR-25, miR-15b, let-7g, miR-542-5p, miR-370, miR-325, miR-134, miR-198, miR-721, miR-515-3p, miR-680, miR-601, miR-206, miR-202, miR-671, miR-381, miR-630, miR-759, miR-564, miR-709, miR-513, miR-298, miR-15b, miR-20a, miR-721, miR-30d, miR-320, miR-466a-5p, miR-21, miR-214, miR-575, miR-513p, miR-15b, let-7i, miR-30d, miR-759, miR-513a-5p, miR-584, miR-637, miR-610, miR-762, miR-765, miR-705, let-7f-1, let7d, miR-494, let-7a, miR-23b, miR-191, miR-21, miR-721, miR-295, miR-690, miR-302b, miR-466a-5p, miR-15a, miR-759, miR-202, miR-302, and miR-608; or
(b) an isolated precursor miRNA that increases the level or activity of one or more target miRNAs as described in (a), wherein a decreased target miRNA level relative to normal is indicative of an airway disease or increased risk of exacerbation of an airway disease; or
(c) an isolated anti-miRNA that decreases the level or activity of one or more target miRNAs as described in (a), wherein an increased target miRNA level relative to normal is indicative of an airway disease or increased risk of exacerbation of an airway disease; or
(d) a kit for determining an miRNA expression level in a sample, comprising the probe array of (a) and instructions for carrying out the determination of miRNA expression level in the sample.

14. The composition of claim 13, wherein the probe array of (a) further comprises a solid support with the plurality of probes attached thereto.
